(19) United States
(12) Patent Application Publication
(10) Pub. No.: US 2013/0209446 A1
(43) Pub. Date: Aug. 15, 2013

(54) Copy Number Variant-Dependent Genes As Diagnostic Tools, Predictive Biomarkers And Therapeutic Targets

(71) Applicants: John D. Shaughnessy, JR., Roland, AR (US); Bart Barlogie, Little Rock, AR (US); Erming Tian, Little Rock, AR (US); Yiming Zhou, Brookline, MA (US)

(72) Inventors: John D. Shaughnessy, JR., Roland, AR (US); Bart Barlogie, Little Rock, AR (US); Erming Tian, Little Rock, AR (US); Yiming Zhou, Brookline, MA (US)

(73) Assignee: Board of Trustees of the University of Arkansas, Little Rock, AR (US)

(21) Appl. No.: 13/810,705

(22) PCT Filed: Jul. 20, 2011
(86) PCT No.: PCT/US11/01286

§ 371 Date: Mar. 6, 2013

Related U.S. Application Data
(60) Provisional application No. 61/456,694, filed on Nov. 10, 2010.
(60) Provisional application No. 61/399,970, filed on Jul. 20, 2010.

(51) Int. Cl. C12Q 1/68; A61K 45/00; A61K 45/06; A61K 31/727

(52) U.S. Cl. CPC C12Q 1/6886; A61K 31/727; A61K 45/00; A61K 45/06
USPC 424/130.1; 506/9; 514/44 R; 514/564; 514/34; 514/110; 514/27; 514/323; 514/56

(57) ABSTRACT

Provided herein are methods of diagnosing and/or treating malignant or pre-malignant conditions in a subject. Overexpression of copy number variant-dependent genes, e.g., genes encoding a cell surface receptor, resulting from copy number changes compared to control is diagnostic of the condition, such as multiple myeloma or monoclonal gammopathy of undetermined significance. Also provided are methods for treating malignant conditions, such as multiple myeloma or a hyperdiploid subtype, with therapeutic agents, with or without other anti-cancer drugs, to downrregulate the overexpressed CNV genes and/or up-regulate the underexpressed genes. Furthermore, methods for lowering drug resistance in multiple myeloma cells via inhibition of platelet activation or thrombin release and for increasing survivability of a multiple myeloma subject via lithibition of PSMD4 gene to increase b-catenin protein expression are provided.

### CROSS-REFERENCE TO RELATED APPLICATIONS

This international application claims benefit of priority under 35 U.S.C. §119(e) of provisional application U.S. Ser. No. 61/456,694, filed Nov. 10, 2010, now abandoned, and U.S. Ser. No. 61/399,970, filed Jul. 20, 2010, now abandoned, the entirety of both of which are hereby incorporated by referenced.

### BACKGROUND OF THE INVENTION

1. Field of the Invention

The present invention generally relates to the field of cancer diagnostics, prognostics, therapeutics, and drug resistance. More specifically, the present invention relates to copy number variant-dependent (CNV) genes, particularly CNV genes encoding a membrane receptor protein as diagnostic tools, predictive biomarkers and therapeutic targets in malignant and pre-malignant pathophysiological conditions, such as multiple myeloma and monoclonal gammopathy of undetermined significance.

2. Description of the Related Art

Multiple myeloma is a malignancy involving an uncontainable clonal expansion of malignant plasma cells in the bone marrow. The malignant plasma cells home to and expand in the bone marrow causing anemia and immunosuppression due to loss of normal hematopoiesis. A hallmark of multiple myeloma is uncontrollable growth in the bone marrow of plasma cells that secrete constant high levels of a paraprotein, causing symptoms of immunodeficiency, anemia, thrombocytopenia, leucopenia, and osteolytic lesions (bone pain) due to loss of normal hematopoiesis. With successful courses of treatment, clinical remissions have been achieved in most cases, but recurrence of myeloma tumor cells is still the obstacle to long-term survival. Poor prognosis is frequently associated with significant cytogenetic anomalies and increased drug resistance at relapsed stages.

The concept of myeloma stem cells is based on the theory that plasmablasts give rise to myeloma cells via constant clonogenic reproduction and differentiation (1). In previous studies, the “myeloma stem cell” referred to a subset of B cells with the CD138−/CD20+/CD27+ phenotype coexisting with the majority of terminally differentiated myeloma cells. By depleting a large portion of CD138+/CD19− myeloma cells, a small number of CD138−/CD19+ cells remained. These cells proliferated with high efficiency, giving rise to progeny with strong CD138 expression (CD138++) both in vitro and in vivo. By contrast, the counterpart of terminally differentiated CD138++ myeloma cells had limited or no clonal potential to recapitulate growth (2). The researchers also found that identical cytogenetic aberrations were maintained indefinitely throughout the replications. Nevertheless, the developmental path of the myeloma stem cells to its fully differentiated progeny is still unknown; more intriguingly, the mechanism that modulates myeloma cell maturation in vitro has yet to be elucidated.

Despite high-dose chemotherapies, the “stem-ness” of these latent malignant cells prevents the disease from being eradicated. Research toward the identification of the “myeloma stem cell” has focused on a subset of B cells that have a low proliferation index and self-renewal properties similar to healthy hematopoietic stem cells, but which constantly reproduce tumor progeny. These so-called myeloma stem cells were found not only in myeloma bone marrow, but also in established human myeloma cell lines (HMCLs). The evidence showed that this subset of cells present CD138−/CD20+/CD27+ surface markers and initiate clonal expansion in cell cultures and tumor growth in immune-deficient mice. By comparison, the vast majority of syndecan-1 positive (CD138++) cells failed to repopulate both in vitro and in vivo.

Protease-activated receptors (PARs, also known as thrombin receptors) are G-protein-coupled receptors, activated by cleavage of their N-terminal domains by serine proteases. Four subtypes of receptors have so far been cloned (PAR1-4). PAR1, also known as coagulation factor II (F2R), is the most characterized member of this receptor family and is activated by the endogenous serine protease thrombin. Thrombin-mediated PAR1 activation induces platelet aggregation.

Activation of PAR1 by thrombin stimulates von Willebrand factor release, tissue factor expression and adhesion molecule expression, which further promotes clotting and coagulation as well as facilitating the rapid adherence of neutrophils, monocytes and lymphocytes to endothelial cells. Thrombin has direct promitogenic activity in fibroblasts, vascular smooth muscle cells, endothelial cells and some myeloid cells. Thrombin-mediated PAR1 activation also induces expression of promitogenic factors and their receptors such as PDGF/PDGFR and ET-1/ETA and ET-B. PAR1 is known to couple to several heterotrimeric G proteins and regulates multiple kinase signaling pathways including PI 3-K, Src family tyrosine kinases, JNK, Rho kinases, JAK2 and FAK.

There is clear evidence that expression of PAR1 and DKK1, are co-expressed in HY disease. Recent studies indicate that platelets are the primary source of DKK1 in multiple myeloma and that whole genome sequencing in multiple myeloma revealed clustering of mutations in genes that regulate the thromboembolytic pathway, suggesting that inappropriate PAR1-mediated thrombin signaling and suppression of Wnt/b-catenin converge to contribute to the pathophysiology of hyperdiploid multiple myeloma. Moreover, PAR1 maps to chromosome 5q13 and chromosome 5 gains are frequent in multiple myeloma. A cardinal feature of HY disease is trisomies of chromosomes 3, 5, 7, 9, 11, 15, 19 and 21. However, to date, there is no definitive understanding as to how PAR1 is linked to multiple myeloma disease.

Experimental evidence suggests that PAR1 can induce numerous cell phenotypes, i.e., proliferation, differentiation (3). These differences are likely dependent on the type of G protein being expressed by the cells. In the HY subtype of multiple myeloma, GEP shows the overexpression of GNG11, a potent inducer of cell senescence, i.e., quiescence (4). Disease relapse often occurs at sites of focal lesions, suggesting that latent tumor cells reside in these sites.

There is a recognized need in the art for improved methods for diagnosing, treating and inhibiting or preventing drug resistance or quiescence in multiple myeloma. The prior art is deficient in methods for utilizing copy number variant-dependent genes, such as PAR1, their resultant products and signaling pathways as diagnostic and predictive biomarkers and/or therapeutic markers in malignant and pre-malignant conditions. The present invention fulfills this long-standing need in the art.

### SUMMARY OF THE INVENTION

The present invention is directed to a method for diagnosing a malignant or premalignant pathophysiological condition in a subject. The method comprises obtaining a biological sample from the subject and determining the expression levels of copy number variant-dependent (CNV) genes associated with the cancer in the sample, where the genes encode a cell surface receptor protein. The expression levels of the CNV genes in the sample are compared with the expression levels of CNV genes in a control sample; wherein one or both of an overexpression or an underexpression of CNV genes as a result of copy number changes compared to control is diagnostic of the pathophysiological condition. In a related method the diagnostic CNV genes are a therapeutic target for a malignant condition and the method further comprises administering a pharmacological amount of at least one therapeutic agent effective to downregulate one or more of the overexpressed CNV genes or to upregulate one or more of underexpressed genes or gene products or a combination thereof, where altering the expression of the one or more genes or gene products treats the malignant condition.

The present invention also is directed to a method for diagnosing a multiple myeloma in a subject. The method comprises obtaining a bone marrow sample from the subject and determining the expression levels of one or more copy number variant-dependent (CNV) genes PAR1, IL6R, IGF2R, GPR89A, EPHB1, GPR180, IL10RB, EGFR, DDR2, CCRL2, ADRB2, ADORA2A, GPR137B, CHRNA5, S1PR3, GPR146, GABRB3, PAQR6, HMMR, PTPRN2, MET, ADIPOR2, NCR1/p46NK, GPR175 or NPR3 in plasma cells comprising the bone marrow sample. The expression levels of the one or more of genes in the subject sample are compared with expression levels of the genes in a control sample; wherein one or both of an overexpression or an underexpression of the genes in the subject sample as a result of copy number changes compared to the control sample is diagnostic of the multiple myeloma. In a related method the diagnostic CNV genes are a therapeutic target for multiple myeloma and the method further comprises administering a pharmacological amount of at least one therapeutic agent effective to downregulate one or more of the overexpressed genes or to upregulate one or more of underexpressed genes or gene products or a combination thereof, where altering the expression of the one or more genes or gene products treats the malignant condition.

The present invention is directed further to a method for diagnosing a hyperdiploid myeloma subtype in a subject. The method comprises obtaining a bone marrow sample from the subject and determining an expression level or copy number of PAR1 gene and expression levels of one or more of PAR2, PAR3 or PAR4 genes in plasma cells comprising the bone marrow sample. An overexpression or increased copy number of PAR-1 and underexpression of one or more of PAR2, PAR3 or PAR4 compared to a control sample is diagnostic of the multiple myeloma.

The present invention is directed further still to a method for treating a cancer in a subject. The method comprises administering a pharmacological amount of at least one therapeutic agent effective to downregulate one or more overexpressed copy number variant-dependent (CNV) genes or gene products or to upregulate one or more underexpressed genes or gene products or a combination thereof where the genes are associated with the cancer. Altering expression of the one or more genes or gene products treats the cancer. In a related method PAR1 is downregulated and one or more of PAR2, PAR3 or PAR4 are upregulated in a multiple myeloma and the method further comprises simultaneously administering a pharmacological amount of another therapeutic agent effective to inhibit DKK1 signaling.

The present invention is directed further still to a method for treating a multiple myeloma in a subject. The method comprises administering a pharmacological amount of a therapeutic agent effective to inhibit the expression of one or more copy number variant-dependent (CNV) genes or gene products thereof, said genes comprising PAR1, IL6R, IGF2R, GPR89A, EPHB1, GPR180, IL10RB, EGFR, DDR2, CCRL2, ADRB2, ADORA2A, GPR137B, CHRNA5, S1PR3, GPR146, GABRB3, PAQR6, HMMR, PTPRN2, MET, ADIPOR2, NCR1/p46NK, GPR175 or NPR3 in either of a membrane or a soluble form, thereby treating the multiple myeloma. In a related method, the CNV gene is PAR1 and the method further comprises simultaneously administering a pharmacological amount of another therapeutic agent effective to inhibit DKK1 signaling.

The present invention is directed further still to a method for treating a multiple myeloma in a subject. The method comprises administering a pharmacological amount of a therapeutic agent effective to downregulate the expression of PAR-1 gene or an activity of a gene product thereof, thereby treating the multiple myeloma. In a related method, a further step comprises administering a pharmacological amount of a therapeutic agent effective to upregulate the expression of PAR2 and PAR3 genes or activities of gene products thereof. In another related method, a further step comprises administering a pharmacological amount of one or more anti-cancer drugs effective to treat the multiple myeloma.

The present invention is directed further still to a method for treating a multiple myeloma in a subject. The method comprises administering an amount of a therapeutic agent pharmacologically effective to reduce megakaryocyte growth, thereby treating the multiple myeloma.

The present invention is directed further still to a method for lowering drug resistance in multiple myeloma cells. The method comprises administering, one or more times, a pharmacological amount of a therapeutic agent effective to inhibit one or both of platelet activation or thrombin release in the multiple myeloma cells, wherein inhibition induces said multiple myeloma cells out of a state of quiescence, thereby lowering drug resistance in the cells.

The present invention is directed further still to a method for increasing survivability of a subject with a multiple myeloma. The method comprises administering, one or more times, a pharmacological amount of a therapeutic agent effective to inhibit PSMD4 gene, wherein inhibition increases expression b-catenin gene or an activity of b-catenin protein, thereby increasing survivability of the subject.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows flow cytometry results showing that PAR1/F2R is present on the cell surface of myeloma cell lines (ARK, H929, INA6) that have high levels of F2R full length ORF by RT-PCR. The F2R positive cells are a subpopulation with weak CD138 (y-axis) and CD38 (x-axis) (upper) when F2R is detected using a monoclonal antibody plus FITC conjugated secondary antibody (lower).

FIGS. 2A-2D depict levels of PAR1/F2R gene expression in various cell lines and tissues. FIGS. 2A-2B show that PAR1/F2R gene expression is highest in the hyperdiploid (HY) subtype compared with the remaining multiple myeloma subtypes, healthy plasma cells (NPC), and established human myeloma cell lines (MMCL). As a comparison, FIGS. 2C-2D show PAR1 expression in various B cells and plasma cells (PC) obtained from bone marrow aspirates (BM), plasma cells or bone biopsies obtained from subjects with Waldenstrom's macroglobulinemia (WM), multiple myeloma (MM), monoclonal gammopathy of undetermined significance (MGUS) or smoldering multiple myeloma (SMM), multiple myeloma cell lines, tonsil tissue, and normal tissues (FIG. 2C) and in various cancer cell lines (FIG. 2D).

FIG. 3 shows a combined aCGH and GEP analysis that indicates PAR1/F2R expression is strongly correlated with gains of chromosome 5 (upper: dark gray=gain, light gray=loss), a consistent feature of the HY molecular subtype of multiple myeloma (lower panel [F2R expression]: dark gray=high, light gray=low).

FIG. 4 shows that PAR1 expression is higher in the cells isolated from myeloma focal lesions using random bone marrow aspirations (RNAS) and fine needle bone marrow aspirations (FNAS).

FIG. 5 shows a combined aCGH and GEP analysis that indicates IL6R expression is strongly correlated with gains of chromosome 1q21 (upper: dark gray=gain, light gray=loss). The lower panel compares IL6R, DKK1 and CCND1 expression with PAR1/F2R expression in the multiple myeloma subtypes: dark gray=high, light gray=low.

FIG. 6 shows the expression levels (MAS5.0 signal) of the four PAR family members in healthy and diseased cells. The samples are as follows: BMBC=CD19 selected B-cells from bone marrow of healthy donors; TBC=CD19 selected B-cells from inflamed tonsil; WMBC=CD19 selected B-cells from bone marrow of patients with Waldenstrom's macroglobulinemia; NPC=CD138-selected plasma cells from bone marrow of healthy donors; SWAS=CD138-selected plasma cells from bone marrow of patients with monoclonal gammopathy of undetermined significance and smoldering myeloma; RNAS=CD138-selected plasma cells from bone marrow of patients with newly diagnosed multiple myeloma; FNAS=CD138-selected plasma cells from bone marrow of patients with newly diagnosed multiple myeloma; samples taken from focal lesions by CT-guided fine needle aspiration as opposed to random sampling of the iliac crest; REFR=CD138-selected plasma cells from bone marrow of patients with relapsed refractory multiple myeloma; MMCL=multiple myeloma cell lines; FTLB=whole bone from human fetus; NBX=whole core bone biopsy from healthy donor; WMBX=whole bone biopsy from patients with Waldenstrom's macroglobulinemia; SWBX=whole bone biopsy from patients with monoclonal gammopathy of undetermined significance and smoldering myeloma; RNBX=whole bone biopsy from patients with newly diagnosed multiple myeloma; FNBX=whole bone biopsy of a focal lesion from patients with newly diagnosed multiple myeloma. Core biopsy taken under CT-guidance; NT=normal tissues. The color of each cell in the tabular image represents the expression level of each gene, with red representing an expression greater than the mean, green representing an expression less than the mean, and the deeper color intensity representing a greater magnitude of deviation from the mean.

FIG. 7 shows the underexpression of GNG11 compared to PAR1/F2R expression in subtypes the multiple myeloma subtypes: dark gray=high, light gray=low.

FIG. 8A-8B show the correlation of DKK1 gene expression (FIG. 8A) and GNG11 gene expression (FIG. 8B) molecularly defined subtype MM and healthy PC and myeloma cell lines.

FIG. 9 shows RT-PCR amplifications of a full-length open reading frame of PAR1/F2R and β-action, as internal control, in HMCLs that demonstrates various levels of PAR1 expression.

FIG. 10 shows flow cytometry results showing that PAR1/F2R is present on the cell surface of myeloma cell lines (OPM2, SKMM1, RPMI8226) that have high levels of F2R full length ORF by RT-PCR. The F2R positive cells are a subpopulation with weak CD138 (y-axis) and CD38 (x-axis) (upper) when F2R is detected using a monoclonal antibody plus FITC conjugated secondary antibody (lower). The intensity of PAR1/F2R cell surface markers in a subset myeloma cell line correlates with the gene expression levels of PAR1/F2R shown in FIG. 4.

FIG. 11 shows flow cytometry results showing that PAR1/F2R is present on the cell surface of MMPC in bone marrow aspirates. The F2R positive cells are a subpopulation with weak CD138 (y-axis) and CD38 (x-axis) (upper) when F2R is detected using a monoclonal antibody plus FITC conjugated secondary antibody (lower).

FIG. 12A-12D shows HMLCs cultured with LOVENOX. The addition of LOVENOX (low molecular weight Heparin) to cell culture media may promote cell proliferation of myeloma cell lines that express high levels of PAR1/F2R.

FIGS. 13A-13B show that in a MTT cell proliferation assay the addition of human thrombin to cell culture media may induce the quiescence of myeloma cell lines that express high levels of PAR1/F2R (ANBL1, H929, and IN6), but not affect F2R negative cell line OPM2.

FIG. 14 shows high expression of F2R in HY disease and high thrombin production by liver may explain the tendency for HY multiple myeloma to metastasize to the liver.

FIG. 15 Is Western blots of the protein fractions from each HMCL shows the distribution of β-catenin in the cytoplasm and nucleus. β-tubulin is for cytoplasmic protein loading control.

FIGS. 16A-16E depict β-catenin accumulation in various cells using immunohistochemistry. In FIG. 16A fluorescent immunohistochemistry stain (FITC) showing β-catenin accumulating at AJs (zip-like bright structures) in epithelial cells (HeLa cell line). In FIG. 16B in myeloma cells, β-catenin is accumulated in the nucleus (DAPI counterstained) and evenly distributed in the cytoplasm but does not construct AJs (JJN3 cell line). In FIG. 16C β-catenin is translocated to the cytoplasm during mitosis (JJN3 cell line). In FIG. 16D At the end of mitosis, β-catenin is evenly distributed into the daughter cells and relocates back to the nuclei (JJN3 cell line). In FIG. 16E in co-culture, myeloma cells (JJN3) attach to stromal cells (HS-5), with AJs between the two types of cells.

### DETAILED DESCRIPTION OF THE INVENTION

As used herein, the following terms and phrases shall have the meanings set forth below. Unless defined otherwise, all technical and scientific terms used herein have the same meaning as commonly understood to one of ordinary skill in the art.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. The terms “comprise” and “comprising” are used in the inclusive, open sense, meaning that additional elements may be included.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “malignant condition” refers to a pathophysiological condition, particularly a cancer, that is characterized by a tendency to become progressively worse and to potentially result in death even with treatment. A malignant condition, such as can be characterized by invasiveness, metastasis to other tissues and uncontrolled proliferation of malignant cells.

As used herein, the term “pre-malignant” refers to pathophysiological condition that while initially non-malignant or benign, may progress or transform to a malignant condition, as defined herein, over a period of time. For example, about 1% of monoclonal gammopathy of undetermined significance (MGUS) conditions per year can transform to multiple myeloma.

As used herein, the terms “control” or “control sample” refer to a sample, e.g., of bone marrow or plasma cells, obtained from a healthy individual, or to a cell line maintained in vitro on which a procedure or assay is performed, e.g., global gene expression profiling (GEP), fluorescent in situ hybridization (FISH), DNA isolation and array-based comparative genomic hybridization (aCGH), MTT cell proliferation assays, etc., to provide a baseline or normal result to which samples of interest are compared.

As used herein, the terms “overexpression” or “underexpression” refer to a quantifiable level of gene expression, such as amount of mRNA, protein or other gene product, copy number, etc. that is greater or lesser, respectively, than a corresponding standard, norm or control level of expression or copy number.

As used herein, the terms “up-regulate” or “down-regulate” refers to increasing or decreasing, respectively, an expression level of a gene. Thus, an up-regulated or down-regulated gene is one which has been observed to have a higher or lower expression level, respectively, for example, as determined by higher or lower mRNA levels, compared to a control sample.

As used herein, the phrase “pharmacological amount” refers to an amount of a therapeutic agent, chemotherapeutic agent, anti-cancer drug, immunomodulatory drug, etc. that elicits a therapeutic response to a pathophysiological condition, for example, but not limited to, a cancer, such as multiple myeloma, when administered to a subject having the condition. Determination of pharmacological amounts are well-within the purview of one of ordinary skill in the art.

As used herein the term “copy number variant-dependent genes” refers to genes whose expression in a malignant condition, for example, multiple myeloma, is correlated with copy number changes.

As used herein, the terms “subject”, “individual” or “patient” refers to a mammal, preferably a human, who has, is suspected of having or at risk for having a malignant or pre-malignant pathophysiological condition, for example, but not limited to, a multiple myeloma, a subtype of multiple myeloma or monoclonal gammopathy of undetermined significance.

In one embodiment of the present invention there is provided a method for diagnosing a malignant or premalignant pathophysiological condition in a subject, comprising the steps of obtaining a biological sample from the subject; determining the expression levels of copy number variant-dependent (CNV) genes associated with the cancer in the sample, said genes encoding a cell surface receptor protein; and comparing the expression levels of the CNV genes in the sample with expression levels of CNV genes in a control sample; wherein one or both of an overexpression or an underexpression of CNV genes as a result of copy number changes compared to control is diagnostic of the pathophysiological condition.

Further to this embodiment the diagnostic genes are a therapeutic target for a malignant condition where the method further comprises administering a pharmacological amount of at least one therapeutic agent effective to downregulate one or more of the overexpressed CNV genes or gene products or to upregulate one or more of underexpressed genes or gene products or a combination thereof, wherein altering expression of the one or more genes or gene products treats the multiple myeloma. In another further embodiment PAR1 is downregulated in a multiple myeloma and the method further comprises simultaneously inhibiting DKK1 signaling with another therapeutic agent.

In all embodiments the therapeutic agents may be a nucleic acid, a small molecule inhibitor or an antibody. Also, the malignant condition may be a multiple myeloma and the pre-malignant condition may be monoclonal gammopathy of undetermined significance. Particularly, the multiple myeloma may be a molecular subtype defined as hyperdiploid myeloma (HY). In addition the biological sample may be bone marrow or plasma cells.

Also in all embodiments the CNV genes may be PAR1, IL6R, IGF2R, GPR89A, EPHB1, GPR180, IL10RB, EGFR, DDR2, CCRL2, ADRB2, ADORA2A, GPR137B, CHRNA5, S1PR3, GPR146, GABRB3, PAQR6, HMMR, PTPRN2, MET, ADIPOR2, NCR1/p46NK, GPR175 or NPR3. In one aspect of these embodiments the overexpressed genes may be PAR1 or IL6R. In a related aspect, PAR1 may overexpressed and one or more of PAR2, PAR3 or PAR4 are underexpressed. In both aspects the overexpression of PAR1 may result from a trisomy of PAR1.

In another embodiment of the present invention there is provided a method for diagnosing a multiple myeloma in a subject, comprising the steps of obtaining a bone marrow sample from the subject; determining the expression levels of one or more copy number variant-dependent (CNV) genes PAR1, IL6R, IGF2R, GPR89A, EPHB1, GPR180, IL10RB, EGFR, DDR2, CCRL2, ADRB2, ADORA2A, GPR137B, CHRNA5, S1PR3, GPR146, GABRB3, PAQR6, HMMR, PTPRN2, MET, ADIPOR2, NCR1/p46NK, GPR175 or NPR3 in plasma cells comprising the bone marrow sample; and comparing the expression levels of the one or more of genes in the subject sample with expression levels of the genes in a control sample; wherein one or both of an overexpression or an underexpression of the genes in the subject sample as a result of copy number changes compared to the control sample is diagnostic of the multiple myeloma.

Further to this embodiment the diagnostic genes are a therapeutic target for multiple myeloma where the method further comprises administering a pharmacological amount of at least one therapeutic agent effective to downregulate one or more of the overexpressed genes or gene products or to upregulate one or more of underexpressed genes or gene products or a combination thereof, wherein altering expression of the one or more genes or gene products treats the multiple myeloma. In another further embodiment PAR1 is downregulated and the method further comprises simultaneously inhibiting DKK1 signaling with another therapeutic agent.

In all embodiments the therapeutic agents may be a nucleic acid, a small molecule inhibitor or an antibody. Also, the multiple myeloma may be a molecular subtype defined as hyperdiploid myeloma (HY). In one aspect of these embodiments the overexpressed genes may be PAR1 or IL6R. In a related aspect, PAR1 may overexpressed and one or more of PAR2, PAR3 or PAR4 are underexpressed. In both aspects the overexpression of PAR1 may result from a trisomy of PAR1.

In yet another embodiment of the present invention there is provided a method for diagnosing hyperdiploid myeloma subtype in a subject, comprising the steps of obtaining a bone marrow sample from the subject; and determining an expression level or copy number of PAR1 gene and expression levels of one or more of PAR2, PAR3 or PAR4 genes in plasma cells comprising the bone marrow sample; wherein overexpression or increased copy number of PAR-1 and underexpression of one or more of PAR2, PAR3 or PAR4 compared to a control sample is diagnostic of the multiple myeloma. In this embodiment the increased copy number of PAR1 may be a trisomy of PAR1.

In yet another embodiment of the present invention there is provided a method for treating a cancer in a subject, comprising the step of administering a pharmacological amount of at least one therapeutic agent effective to downregulate one or more overexpressed copy number variant-dependent (CNV) genes or gene products or to upregulate one or more underexpressed genes or gene products or a combination thereof, where the genes are associated with the cancer and where altering expression of the one or more genes or gene products treats the cancer. Further to this embodiment PAR1 is downregulated and one or more of PAR2, PAR3 or PAR4 are upregulated in a multiple myeloma and the method comprises simultaneously administering a pharmacological amount of another therapeutic agent effective to inhibit DKK1 signaling.

In both embodiments the therapeutic agent may be a nucleic acid, a small molecule inhibitor or an antibody. Also, the cancer may be a multiple myeloma. Particularly, the multiple myeloma may be a molecular subtype defined as hyperdiploid myeloma (HY). Also, in both embodiments the CNV genes may be PAR1, IL6R, IGF2R, GPR89A, EPHB1, GPR180, IL10RB, EGFR, DDR2, CCRL2, ADRB2, ADORA2A, GPR137B, CHRNA5, S1PR3, GPR146, GABRB3, PAQR6, HMMR, PTPRN2, MET, ADIPOR2, NCR1/p46NK, GPR175 or NPR3. In one aspect the overexpressed CNV genes are PAR1 or IL6R. In a related aspect the overexpressed CNV gene is PAR1 and the underexpressed genes are PAR2 or PAR3.

In yet another embodiment of the present invention there is provided a method for treating a multiple myeloma in a subject, comprising the step of administering a pharmacological amount of a therapeutic agent effective to inhibit the expression of one or more copy number variant-dependent (CNV) genes or gene products thereof, said genes comprising PAR1, IL6R, IGF2R, GPR89A, EPHB1, GPR180, IL10RB, EGFR, DDR2, CCRL2, ADRB2, ADORA2A, GPR137B, CHRNA5, S1PR3, GPR146, GABRB3, PAQR6, HMMR, PTPRN2, MET, ADIPOR2, NCR1/p46NK, GPR175 or NPR3 in either of a membrane or a soluble form, thereby treating the multiple myeloma. Further to this embodiment the CNV gene is PAR1, the method Comprising simultaneously administering a pharmacological amount of another therapeutic agent effective to inhibit DKK1 signaling. In both embodiments the therapeutic agent and the molecular subtype of multiple myeloma are as described supra.

In yet another embodiment of the present invention there is provided a method for treating a multiple myeloma in a subject, comprising the step of administering a pharmacological amount of a therapeutic agent effective to downregulate the expression of PAR-1 gene or an activity of a gene product thereof, thereby treating the multiple myeloma.

In yet another embodiment of the present invention there is provided a method for treating a multiple myeloma in a subject, comprising the step of administering an amount of a therapeutic agent pharmacologically effective to reduce megakaryocyte growth, thereby treating the multiple myeloma.

Further to this embodiment the method comprises administering a pharmacological amount of a therapeutic agent effective to upregulate the expression of one or more of PAR2, PAR3 or PAR4 genes or activities of gene products thereof. In another further embodiment the method comprises administering a pharmacological amount of one or more anti-cancer drugs effective to treat the multiple myeloma. In all embodiments the therapeutic agent may be a nucleic acid, a small molecule or an antibody and the anti-cancer drug may be melphalan, doxorubicin, cytoxan, etoposide, an IMiD, a proteasome inhibitor, or an HDAC inhibitor. Also, the multiple myeloma and subtype thereof are as described supra.

In this embodiment the therapeutic agent may be an inhibitor of expression or activity of one or both of MRVI1 or DKK1 gene or gene product. For example the therapeutic agent may be thalidomide, lenalidomide, or a structural or functional derivative or an analogue thereof. Particularly, the analogue is an ImiD. Also, the multiple myeloma and subtype thereof are as described supra.

In yet another embodiment of the present invention there is provided a method for lowering drug resistance in multiple myeloma cells, comprising the step of administering, one or more times, a pharmacological amount of a therapeutic agent effective to inhibit one or both of platelet activation or thrombin release in the multiple myeloma cells, wherein inhibition induces said multiple myeloma cells out of a state of quiescence, thereby lowering drug resistance in the cells.

In this embodiment the therapeutic agent may be an inhibitor of one or both of expression or activity of PAR1, GNG11, DKK1, or Wnt-β-catenin. For example, the therapeutic agent may be a nucleic acid, a small molecule inhibitor or an antibody. Also, the multiple myeloma and subtype thereof are as described supra.

In yet another embodiment of the present invention there is provided a method for increasing survivability of a subject with a multiple myeloma, comprising the step administering, one or more times, a pharmacological amount of a therapeutic agent effective to increase expression of β-catenin gene or an activity of β-catenin protein, thereby increasing survivability of the subject. The therapeutic agents and the molecular subtype of multiple myeloma are as described supra.

Provided herein are diagnostic and therapeutic methods for malignant and pre-malignant conditions based on overexpression and copy number changes of copy number variant-dependent (CNV)-dependent genes associated with the condition. Preferably, the CNV genes encode a cell surface receptor on cells associated with the condition. In a non-limiting example the copy number variant-dependent genes listed in Table 1 provide a diagnostic model for a multiple myeloma, for example, a hyperdiploid multiple myeloma and monoclonal gammopathy of undetermined significance (MGUS). Particularly, it is demonstrated herein that PAR1 is a copy number sensitive gene, coding for a plasma membrane bound signaling receptor, whose expression is significantly upregulated in myeloma with gains of chromosome 5q. Therefore, the detection of overexpression of PAR1 and copy number changes, such as trisomy of PAR1, represents a viable diagnostic tool, predictive biomarker and therapeutic target in multiple myeloma. Furthermore, it is demonstrated that co-targeting DKK1 and PAR1 in HY type multiple myeloma, may have synergistic effects. Alternatively, IL6R overexpression is useful in the diagnosis of the conditions described herein and is another therapeutic target.

More particularly, in human myeloma cell lines (HMCLs) and myeloma bone marrow, a subset of cells are identified that are positive for PAR1 (coagulation factor II [thrombin] receptor, or F2R) and have weak CD138 and CD38 surface markers. In primary multiple myeloma, PAR1 gene expression is elevated in 50% of cases and is correlated with the predominant genotype of hyperdiploid (HY) myeloma. The PAR1-positive phenotype defines a distinct subpopulation in heterogeneous bone marrow cells and, to a greater degree, in a homogenous myeloma cell line.

Significantly, growth inhibition occurs when myeloma cells that express high levels of PAR1 are exposed to thrombin. Studies of PAR1 functions indicate it is not only a thrombin receptor, but also modulates the phosphorylation of β-catenin, either parallel to or competing with the well-known Wnt pathway. The destination of phosphorylated β-catenin is crucial to the formation of adherens junctions (AJs) at the cell membrane and for gene transcription regulation in the nucleus. It is demonstrated herein that most HMCLs express high levels of β-catenin, which is primarily accumulated in the nucleus, and that resistance to anticancer agents in each cell line is directly linked to the level of β-catenin. Furthermore, thrombin-catalyzed stimulation can enhance the AJs between myeloma cells and stromal cells.

Myeloma cells therefore may be capable of transforming into a quiescent stem-like phenotype that is drug resistant. Particularly, thrombin-induced PAR1 signaling modulates β-catenin redistribution and plays a major role in the reversible transformation of primary myeloma cells to indolent myelomablasts.

Thus, genes identified as copy number variant-dependent genes are at least potential therapeutic targets and, methods of treating malignant conditions, such as, but not limited to, a multiple myeloma or subtype thereof, e.g., hyperdiploid multiple myeloma, are provided herein. Therapeutic agents, such as chemotherapeutic agents, anticancer drugs or other compounds or biomolecules, effective to inhibit or prevent the increase or decrease of expression or increase in copy number of the genes that are diagnostic of a malignant or pre-malignant condition, can inhibit or prevent quiescence of cells associated with acquisition of drug resistance in the condition and can improve the patient's chance for survival. Potential agents may be known in the art, may be synthesized or may be produced via standard chemical synthetic or molecular biological techniques. For example, therapeutic agents may be a nucleic acid, a small molecule or an antibody. Other potential therapeutic agents may be tested via known and standard assays measuring cell proliferation, gene expression and/or copy number levels and/or measuring gene products in cancer cell lines in vitro or in ex vivo samples in the presence or absence of chemotherapeutic agents utilized in known treatment regimens, such as Total Therapy regimens for multiple myeloma.

The therapeutic agents, anticancer drugs, chemotherapeutics or pharmaceutical compositions thereof may be administered independently or in combination one or more times to achieve, maintain or improve upon a therapeutic effect, such as suppression of cell quiescence, inhibition of drug resistance acquisition, or increase in patient survivability. It is well within the skill of an artisan to determine dosage or whether a suitable dosage of either or both of the therapeutic agent and/or anticancer drug comprises a single administered dose or multiple administered doses. An appropriate dosage depends on the subject's health, age, current therapies, the progression or remission of the malignant condition, the route of administration and the formulation used. Formulating a therapeutic agent, anticancer drug, etc. as a pharmaceutical or immunological composition comprising pharmaceutically or immunologically acceptable carriers, adjuvants and/or diluents is well-known in the art.

While the examples provided herein utilize multiple myeloma plasma cells, one of ordinary skill in the art can see that the methods provided herein are readily adapted to other malignant cancers or conditions associated with copy number variant-dependent genes and/or cytogenetic abnormalities. Global gene expression profiling (GEP), fluorescent in situ hybridization (FISH), DNA isolation and array-based comparative genomic hybridization (aCGH), and the statistical analysis techniques provided herein are well-suited to identify genes that are diagnostic of other such conditions and which would provide a diagnostic and/or predictive model and therapeutic targets for treatment and/or prediction of survivability.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Multiple Myeloma Genes with Overexpression Correlating with Copy Number Changes

The Broad Institute/MMRF/MMRC consortium recently reported on the whole genome sequencing of 38 myeloma genomes. Clustering of mutations in components of complement cascade-thromboembolytic cascade in multiple myeloma were examined. Together these data strongly implicate the PAR1 pathway as playing a significant role in myelomagenesis. In a proteomic study, a substantial proportion of multiple myeloma cases exhibited phosphorylation of virtually all signaling molecules in extracts of purified multiple myeloma cells. Given the pleotropic effects of PAR activation, multiple myeloma is strongly correlated with the overexpression of PAR1.

Cells were isolated from bone marrow of newly diagnosed myeloma patients and separated into aliquots with one being subjected to GEP and the other, aCGH, analyses. Copy number variant-dependent (CNV) genes in CD138-selected plasma cells genes were identified and ranked here based on correlation coefficient. Genes whose expression in multiple myeloma correlated with copy number changes in those tumor cells were screened further for plasma membrane receptor genes exhibiting significant correlations with copy number variation of their genomic locus. Table 1 identifies those genes that code for plasma membrane receptors and whose expression is copy-number variant-dependent and upregulated following copy number gains. Genes in Table 1 are ranked based on correlation from highest to lowest values. PAR1 was identified as the highest ranked gene in a list of copy-number variant-dependent genes. PAR1 maps to chromosome 5q13 and expression is highly correlated with gains of chromosome 5 in multiple myeloma. PAR1, also known as coagulation factor II (thrombin) receptor or F2R, is a high affinity receptor for activated thrombin that is coupled to G proteins that stimulate phosphoinositide hydrolysis.

TABLE 1

| | gep:cgh | | | | chromosome |
|---|---|---|---|---|---|
| Affymetrix | correlation | | Entrez | | map |
| ID | coefficient | gene symbol | id | Description | position |
| 203989_x_at | 0.576371069 | F2R/PAR1 | 2149 | G protein coupled receptor; | 5q13 |
| | | | | coagulation factor II | |
| | | | | (thrombin) receptor | |
| 217489_s_at | 0.537431607 | IL6R | 3570 | interleukin 6 receptor | 1q21 |
| 201393_s_at | 0.451020198 | IGF2R | 3482 | insulin-like growth factor 2 | 6q26 |
| | | | | receptor | |
| 220642_x_at | 0.448553928 | GPR89A | 51463 | G protein-coupled receptor | 1q21.1 |
| | | | | 89A | |
| 230425_at | 0.447059932 | EPHB1 | 2047 | EPH receptor B1 | 3q21 |
| 231871_at | 0.425050607 | GPR180 | 160897 | G protein-coupled receptor | 13q32.1 |
| | | | | 180 | |
| 209575_at | 0.417444441 | IL10RB | 3588 | interleukin 10 receptor, beta | 21q22.1 |
| 1565484_x_at | 0.410176459 | EGFR | 1956 | epidermal growth factor | 7p12 |
| | | | | receptor (erythroblastic | |
| | | | | leukemia viral (v-erb-b) | |
| | | | | oncogene homolog, avian) | |
| 205168_at | 0.407903898 | DDR2 | 4921 | discoidin domain receptor | 1q23.3 |
| | | | | tyrosine kinase 2 | |
| 211434_s_at | 0.403914851 | CCRL2 | 9034 | chemokine (C-C motif) | 3p21 |
| | | | | receptor-like 2 | |
| 206170_at | 0.40123865 | ADRB2 | 154 | adrenergic, beta-2-, | 5q31 |
| | | | | receptor, surface | |
| 205013_s_at | 0.375490952 | ADORA2A | 135 | adenosine A2a receptor | 22q11.23 |
| 204137_at | 0.364959719 | GPR137B | 7107 | G protein-coupled receptor | 1q42 |
| | | | | 137B | |
| 206533_at | 0.36289088 | CHRNA5 | 1138 | cholinergic receptor, | 15q24 |
| | | | | nicotinic, alpha 5 | |
| 228176_at | 0.35342551 | S1PR3 | 1903 | sphingosine-1-phosphate | 9q22.1 |
| | | | | receptor 3 | |
| 228770_at | 0.346242311 | GPR146 | 115330 | G protein-coupled receptor | 7p22.3 |
| | | | | 146 | |
| 229724_at | 0.335236082 | GABRB3 | 2562 | gamma-aminobutyric acid | 15q11.2 |
| | | | | (GABA) A receptor, beta 3 | |
| 219236_at | 0.33094356 | PAQR6 | 79957 | progestin and adipoQ | 1q22 |
| | | | | receptor family member VI | |
| 209709_s_at | 0.327155505 | HMMR | 3161 | hyaluronan-mediated motility | 5q33.2 |
| | | | | receptor (RHAMM) | |
| 211534_x_at | 0.32311663 | PTPRN2 | 5799 | protein tyrosine | 7q36 |
| | | | | phosphatase, receptor type, | |
| | | | | N polypeptide 2 | |
| 211599_x_at | 0.323037444 | MET | 4233 | met proto-oncogene | 7q31 |
| | | | | (hepatocyte growth factor | |
| | | | | receptor) | |
| 201346_at | 0.321324962 | ADIPOR2 | 79602 | adiponectin receptor 2 | 12p13.31 |
| 217095_x_at | 0.321133937 | NCR1/p46NK | 9437 | natural cytotoxicity triggering | 19q13.42 |
| | | | | receptor 1 | |
| 218855_at | 0.315780305 | GPR175 | 131601 | G protein-coupled receptor | 3q21.2 |
| | | | | 175 | |
| 219789_at | 0.314005414 | NPR3 | 4883 | natriuretic peptide receptor | 5p14 |
| | | | | C/guanylate cyclase C | |
| | | | | (atrionatriuretic peptide | |
| | | | | receptor C) | |

### Example 2

### Gene Upregulation in Multiple Myeloma and MGUS

### PAR1 is Upregulated in Myeloma Plasma Cells, Focal Osteolytic Lesions

While PAR1 expression is normally low in plasma cells isolated from healthy donors, it progressively increases from the benign MGUS to relapsed multiple myeloma. PAR1 expression is highest in plasma cells isolated from so called focal lesions or medullary plasmacytomas of the bone. Expression of PAR1 is not altered in the related plasma cell malignancy Waldenstrom's macroiglobulinemia. Consistently, Waldenstrom's macroglobulinemia does not exhibit gains of chromosome 5. While PAR1 activation has been shown in cancer, multiple myeloma is the first malignancy where PAR1 activation can be attributed to a genetic lesion, that is, increased copy number of the PAR1 gene. As such, PAR1 signaling may contribute to multiple myeloma disease pathogenesis. It is likely that PAR1 activation primarily occurs via thrombin-mediated cleavage of PAR1.

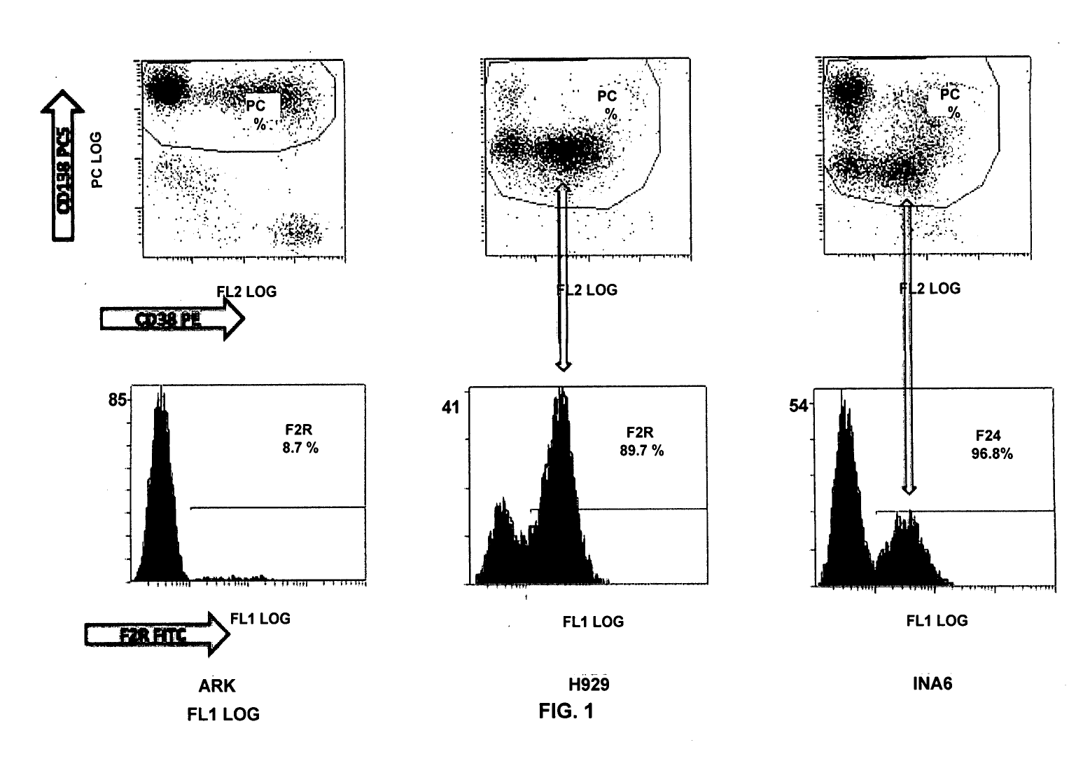

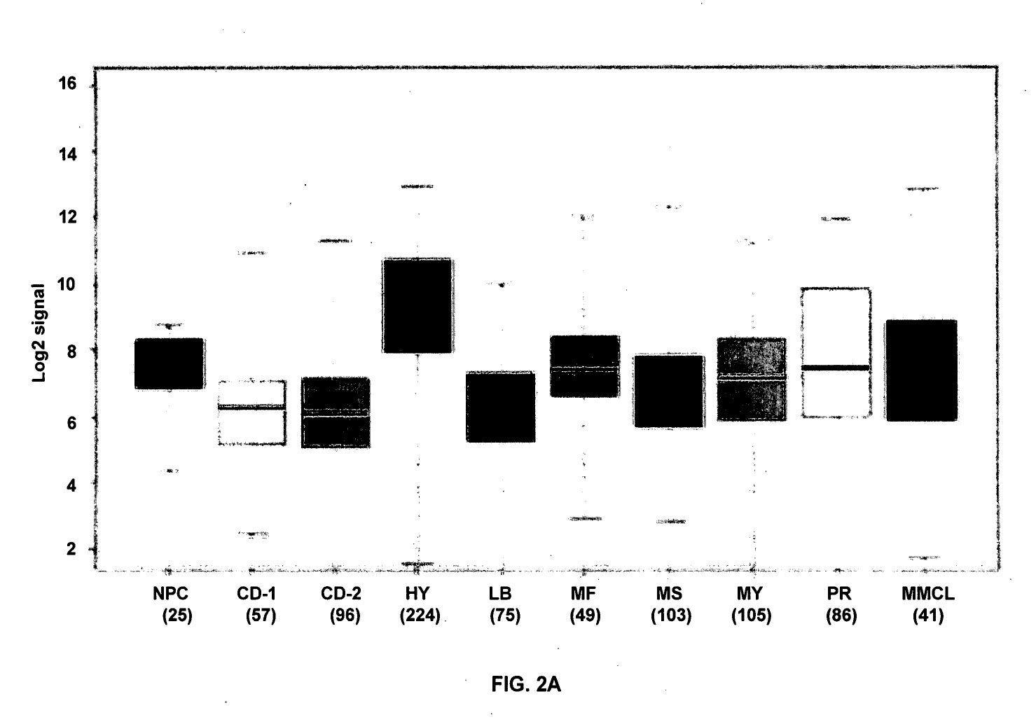

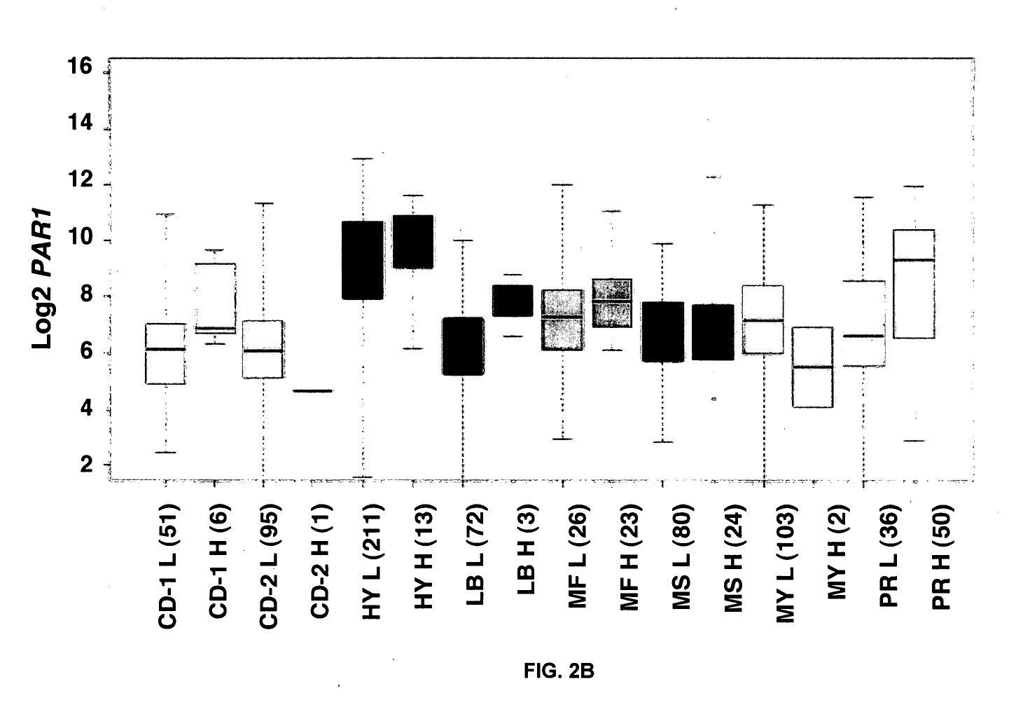

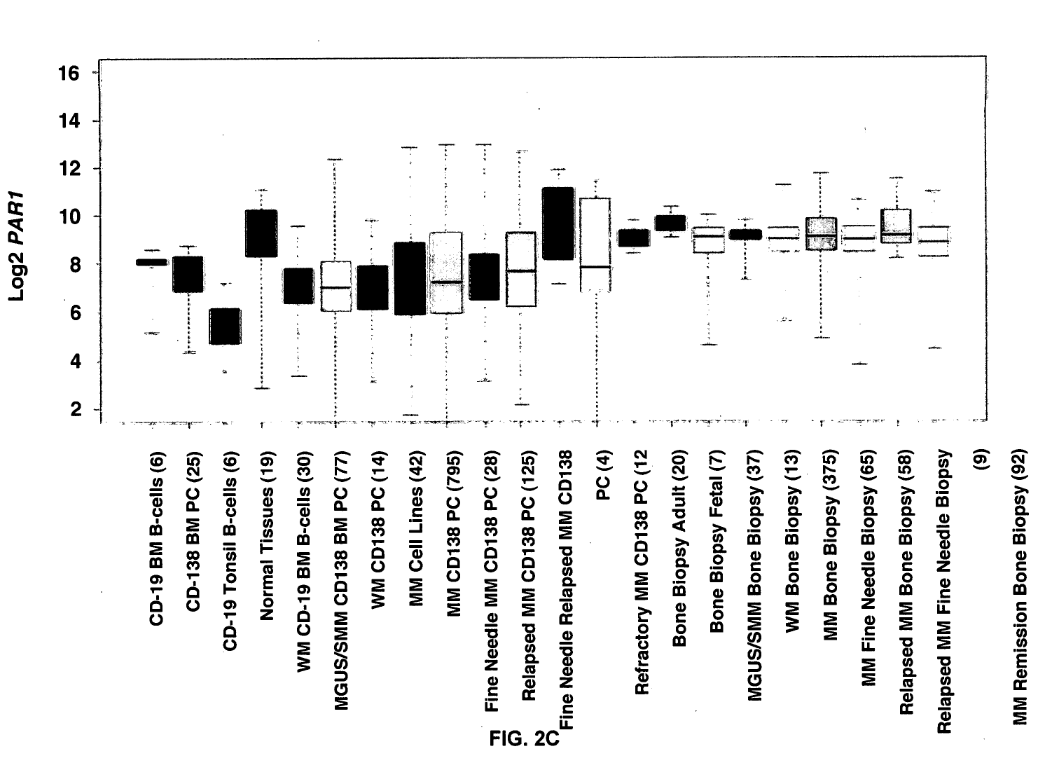

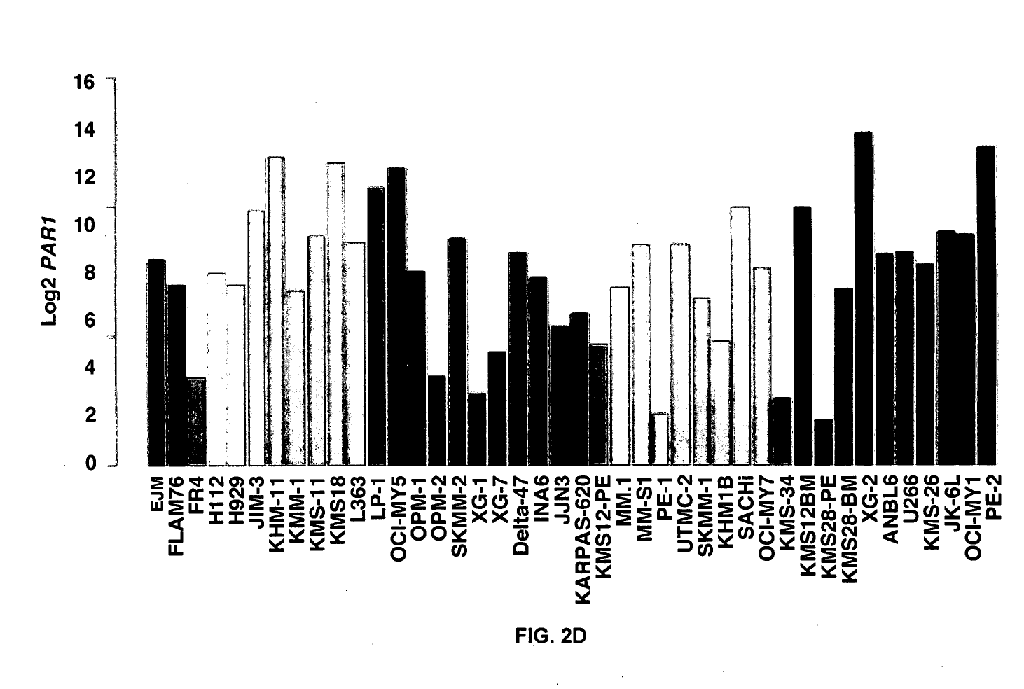

Purified plasma cells are subjected to GEP, aGCH, cytogenetics, and proteomic analyses. PAR1/F2R is present on the cell surface of ARK, H929 and INA6 myeloma cell lines (FIG. 1). The standard prognostic parameters are also well documented for institutional case studies, and GEP-based molecular classification of multiple myeloma subgroups has suggested case-specific therapies (5). By comparing the seven subtypes of multiple myeloma in the Total Therapy II and III protocols, it was determined that PAR1/F2R gene expression is highest (cc=0.57) in the HY subgroup among all the other molecularly defined subtypes (FIGS. 2A-2B). Also, in addition to advanced disease multiple myeloma, PAR1 expression is upregulated in the monoclonal gammopathy of undetermined significance or MGUS stage of the disease at a level intermediate to that seen in newly diagnosed multiple myeloma. and further upregulated in advanced disease. Its chronic activation is likely to drive disease progression in HY disease. Furthermore, PAR1 activation has been shown in other cancers (FIGS. 2C-2D).

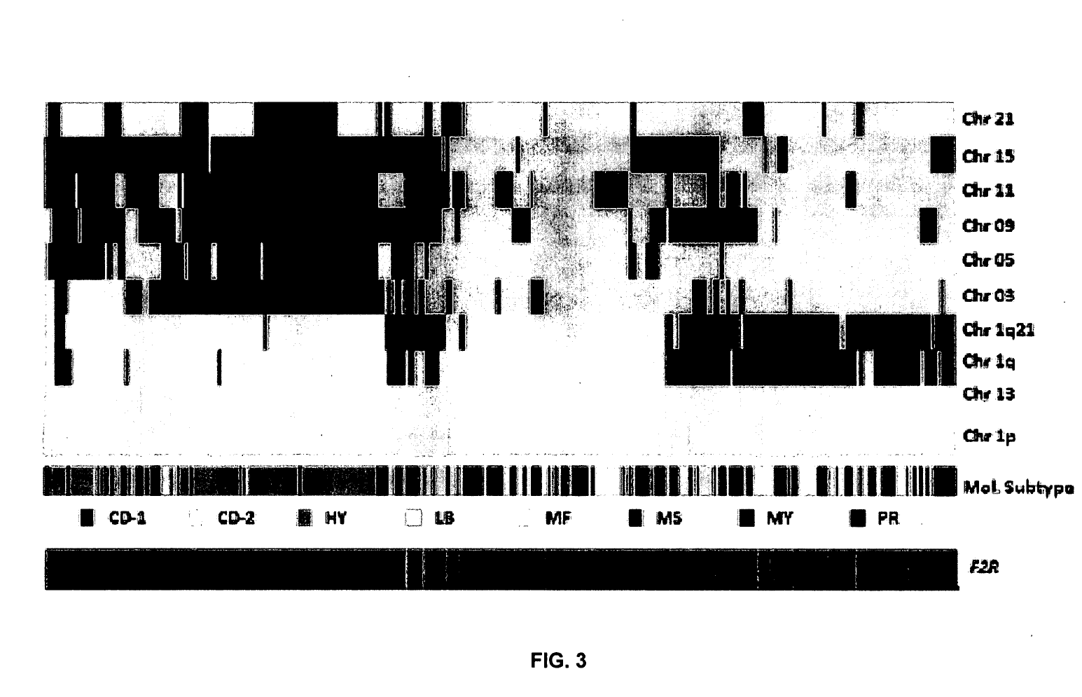

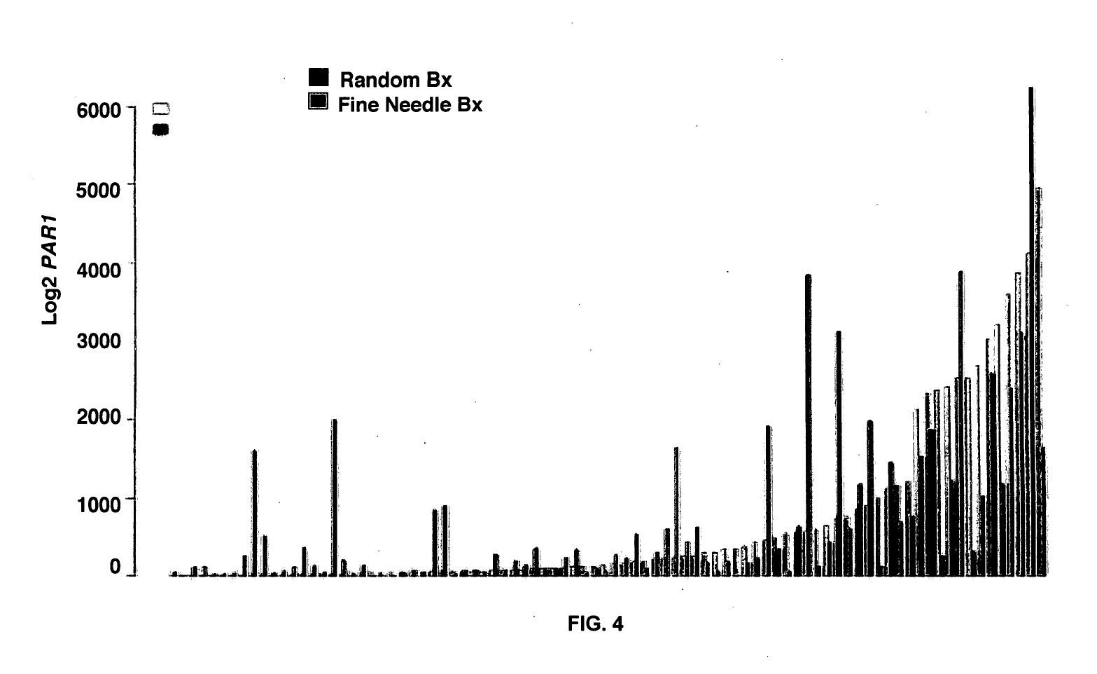

At the time of diagnosis, 224 of 795 patients (˜28%) had the HY subtype. By comparison, PAR1 expression was relatively low in healthy plasma cells and in the HMCLs examined (NPC and MMCL, respectively, in FIG. 2A). By combining paired myeloma aCGH and GEP, it was shown that PAR1 expression is strongly correlated with gains of chromosome 5, a consistent feature of the HY subtype (FIG. 3). GEP of paired bone biopsies (Random Bx) and CT-guided fine needle biopsies (Fine Needle Bx) showed overall higher PAR1 expression in the focal lesions than in bone marrow plasma cells (FIG. 4). PAR1 overexpression occurs in 50% of newly diagnosed multiple myeloma cases, based on more than 3,000 cases examined, and the levels are increased at relapse. Elevated PAR1 expression indicates the progression of primary myeloma cells to the quiescent stem-like stage.

PAR1 expression is highest in CD138+ cells isolated from CT-guided aspirates of MRI-defined focal lesions. Consistent with its ubiquitous expression in many cell types, PAR1 is highly expressed whole bone biopsies across sample types, but consistent with CD138 data it is highest CT-guided fine needle biopsies. Flow cytometry has been used to prove that PAR1 protein is expressed on the cell surface of multiple myeloma cells in a manner consistent with PAR1 mRNA levels. Three color flow cytometry with antibodies to CD138, CD38 and PAR1 indicates that PAR1 is primarily expressed in plasma that are weakly positive for CD138 in both primary disease and MMCL

### IL6R is Upregulated in Multiple Myeloma

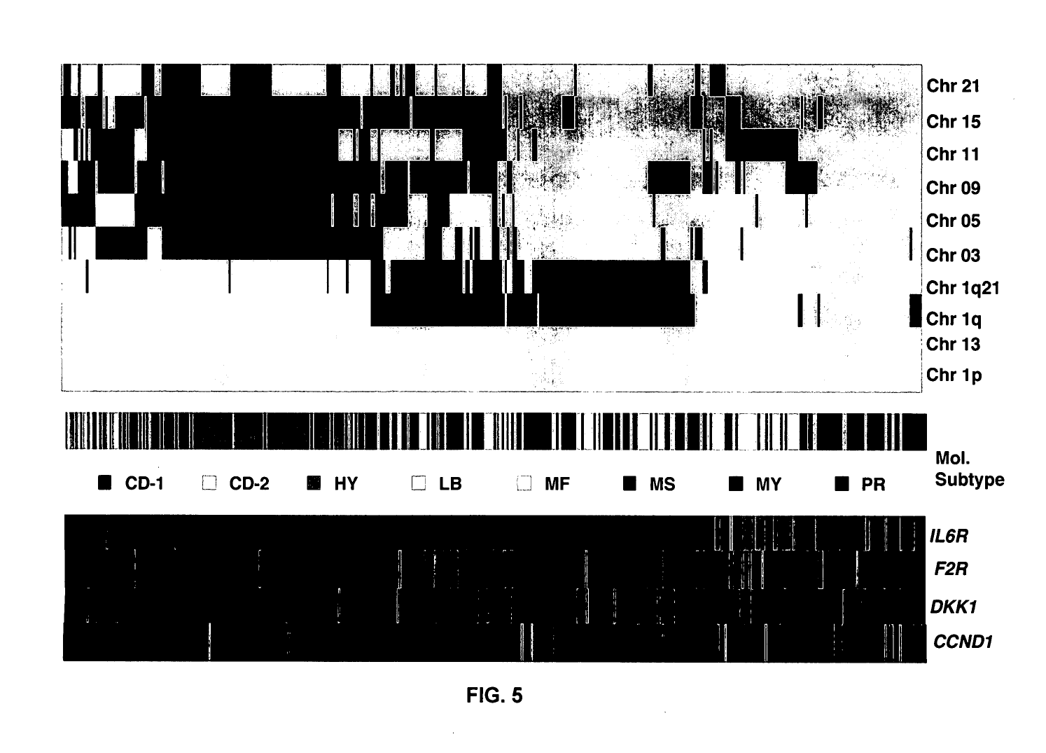

IL6R is the second most significant gene correlated with copy number variant-dependent (cc=0.53) (FIG. 5). PAR1 and IL6R activation are generally mutual exclusive events in multiple myeloma and together account for the great majority of all multiple myeloma cases. The role of IL6 signaling in multiple myeloma is well known, however the significance of PAR1 signaling in multiple myeloma has been unknown. It is also noteworthy that IL6R maps to the 1q21 amplicon in multiple myeloma and its expression levels can define disease with 2, 3 or 4+ copies of 1q21 which in turn is associated with differential prognosis in the disease. It is demonstrated herein that thrombin or plasmin-mediated PAR1 activation could be source of PI3K/Akt activation in multiple myeloma. This is more likely now that whole genome sequencing efforts have failed to show mutations in genes in the PI3K-AKT pathway. Therefore, PAR1 and IL6R represent therapeutic targets in multiple myeloma.

PAR1 is Upregulated while PAR2-PAR4 are Downregulated in Multiple Myeloma and MGUS

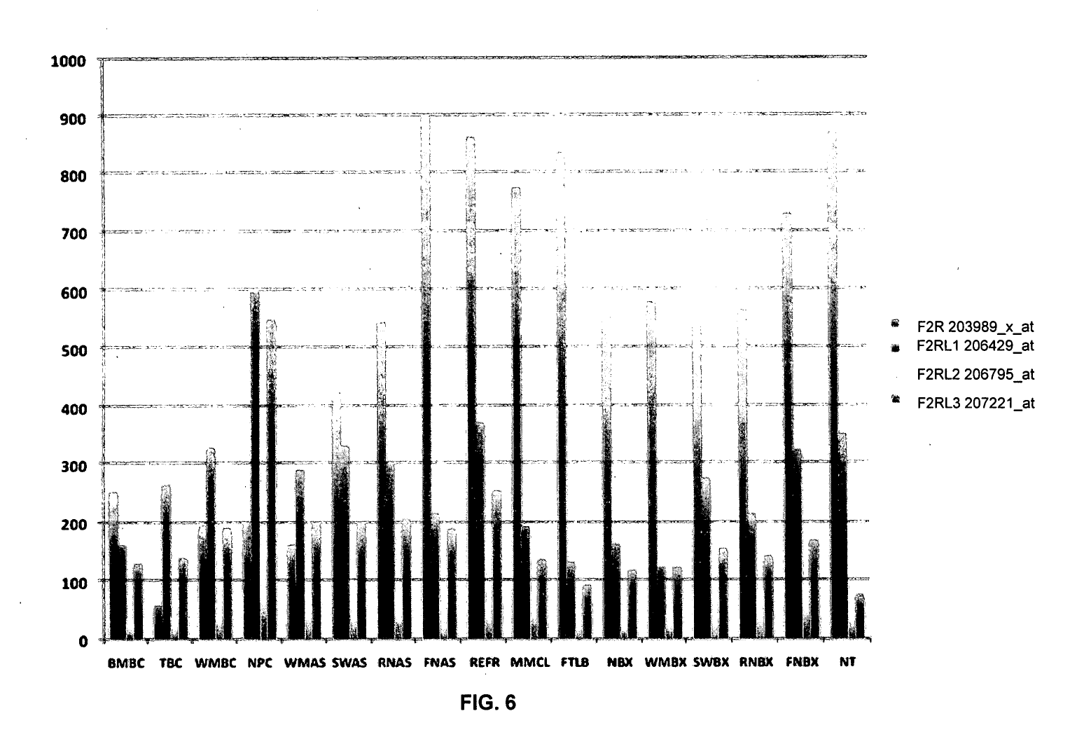

There are 4 PAR1 family members and an analysis of the expression of these four genes in multiple myeloma and other malignant B-cells and their normal counterparts reveals that PAR1 expression is uniquely upregulated in the MGUS and multiple myeloma, particularly, the hyperdiploid subtype, conditions While higher expression is found in CD138 plasma cells from healthy donors, PAR2-PAR4, are downregulated in multiple myeloma (FIG. 6) and also CD138 selected cells from patients with Waldenstrom's macroglobulinemia (WM). There is no evidence of elevated PAR1 expression in Waldenstrom's macroglobulinemia. Thus, in this malignant condition, not only is there a copy-number variant-dependent-driven upregulation of PAR1, there is concomitant downregulation of other PAR1 family members. This may in turn amplify the signal from the copy-number variant-dependent-activated PAR1.

PAR1 Expression Highly Correlates with DKK1 Expression in HY Myeloma

PAR1 expression is highly correlated with DKK1 expression and gains in chromosome 5 in HY disease (FIG. 5). This important link between DKK1 and osteolytic bone loss in multiple myeloma and solid tumor bone metastases has now been established. DKK1 also mobilizes endothelial progenitor cells in the bone marrow and induces a hemorrhage-prone, neo-vasculogenesis in the bone marrow of mice and mesentery of rats (Aicher et al., 2009, Glaw et al., 2010). This vascular phenotype resembles those seen in the eyes of patients with germline loss-of-function mutations in LRP5 or FZD4 causing Osteoporosis-Pseudoglioma and Familial Exudative Vitreoretinopathy. These vascular defects all linked to loss of function of b-catenin during forced vascular development combined with the recent recognition that DKK1 is elevated in most solid tumors, raises the possibility that DKK1 might promote neo-vasculogenesis in the bone marrow of multiple myeloma and cancer metastases.

DKK1 is a secreted inhibitor of Wnt-β-catenin signaling, which is essential for osteoblast differentiation and normal coupled bone turnover. DKK1 overexpression by multiple myeloma cells is abnormal and likely contributes to osteolytic bone disease in multiple myeloma. DKK1 production by multiple myeloma cells might also promote tumor progression by upregulating IL6 in tumor microenvironment. Based on the results related to PAR1 expression, PAR1 expression in DKK1-positive disease may be intimately interconnected. DKK1 might be a direct downstream target of PAR1 activation and DKK1 may facilitate PAR1 signaling. In addition to its role in regulating bone homeostasis, Wnt/b-catenin also plays an important role in modulating immune system development from the primitive haematopoeic stem cell (HSC) and through numerous lineage commitment stages.

DKK1 may also cause a leaky vessel formation in the multiple myeloma bone marrow and contribute to the tumor vasculature in many other cancers. Recent studies have shown that DKK1 is taken up by platelets and that while produced by multiple myeloma cells platelets represent the largest source of DKK1 in multiple myeloma serum. Platelets likely aggregate at sits of vessel leakage, where they release DKK1. It is hypothesized that platelets then release thrombin which in turn cleaves PAR1 leading to its activation. Upon activation, the multiple myeloma cells become quiescent. It is further hypothesized that PAR1-induced quiescence in multiple myeloma primarily occurs downstream of a DKK1-induced angiogenic switch in the bone marrow. A DKK1 mediated angiogenic switch may be relevant to many cancers now known to be DKK1 positive

DKK1-mediated suppression of Wnt-induced osteoblast differentiation contributes to bone disease and also destruction of the hypoxic endosteal niche critical to HSC function. DKK1 induced neovasculogenesis increases the oxygenation in the bone marrow needed to support increased tumor growth/volume. Multiple myeloma typically exhibits two types of growth patterns in the bone. Cells grow in an interstitial, nodular, or a mixed interstitial nodular pattern. Clinical evidence suggests that interstitial growing cells are more sensitive to chemotherapy. MRI-focal lesions often persist even in patients' in whom there is serum immunofixation negativity. Moreover, relapses often occur at sites of focal lesions suggesting that latent tumor cells reside in these sites. Importantly, while the incidence of cytogenetic abnormalities, requiring proliferation of cells in vitro, is comparable, cytogenetic abnormalities in focal lesions does not carry the same dire prognosis as cytogenetic abnormalities is seen in cells from random aspirates.

This may be related to the elevated expression of PAR1 on multiple myeloma tumor cells. It stands to reason that DKK1 mediated damage to the multiple myeloma bone marrow triggers a wound healing response that results in a constitutive activation of platelets, whose thrombin may trigger PAR1 and subsequently DKK1, resulting in a vicious cycle of bone loss and multiple myeloma tumor growth. Platelets loaded with thrombin and DKK1 may be potent inducers of this vicious cycle. Thus, simultaneous neutralization of both DKK1 and PAR1 signaling may have therapeutic value. This theory is supported by the recent finding that megakaryocytes form a multiple myeloma niche within the bone marrow. It is likely that thrombin signaling through PAR1 in multiple myeloma cells forms the basis for this relationship. Furthermore, it is likely that PAR1 activation leads to activation of DKK1. Thus, PAR1 inhibition would suppress DKK1 and therefore suppress bone disease.

The immunomodulatory agents thalidomide and lenalidomide have potent anti-myeloma effects and hyperactivate DKK1 in multiple myeloma cells. These agents are also known to induce life threatening deep-vein thrombosis in multiple myeloma patients and has led to the widespread use of prophylactic use of low molecular weight heparin and other anti-thrombin signaling drugs with their use.

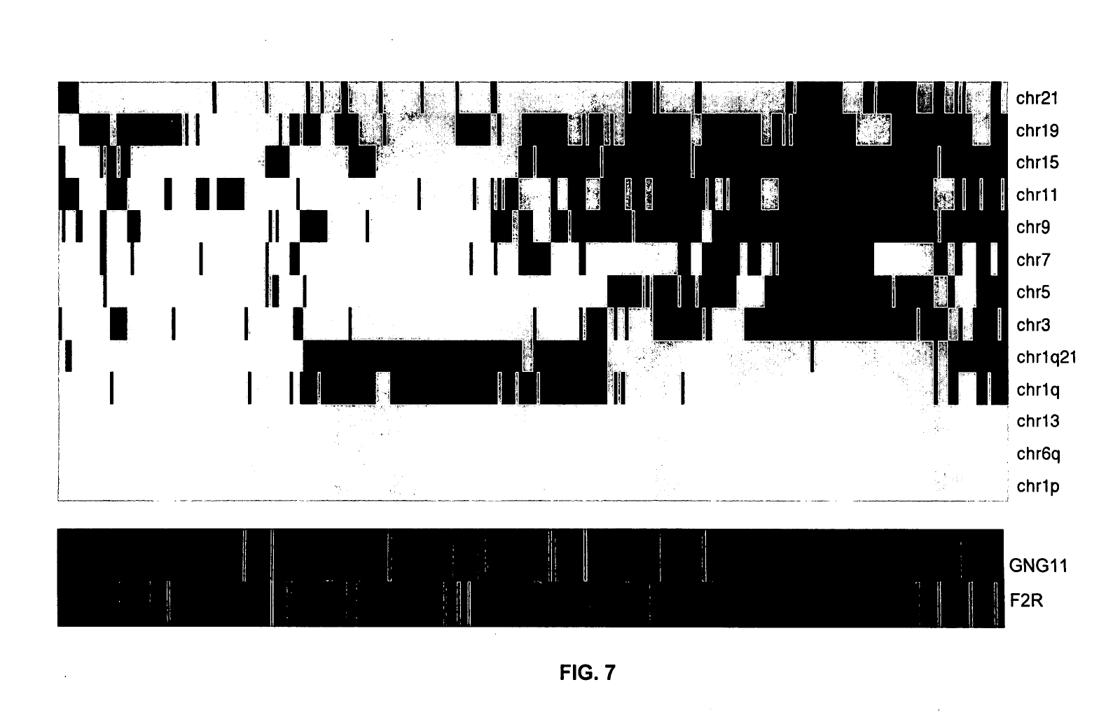

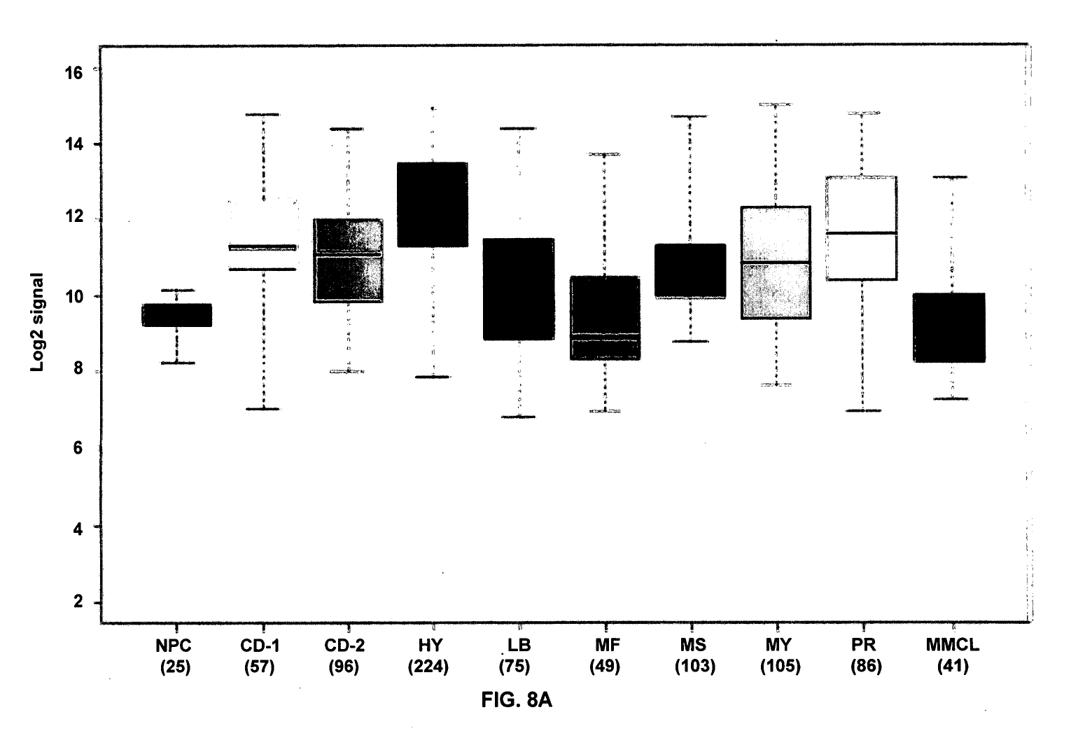

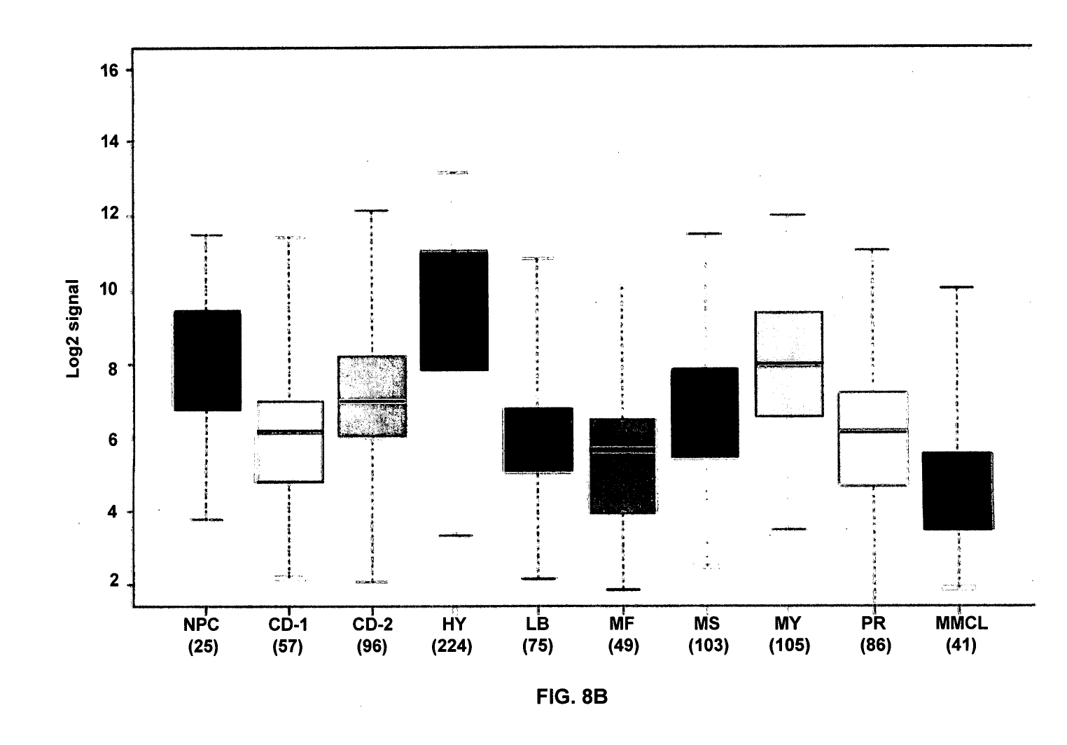

Improved outcomes being witnessed with the use of thalidomide and lenalidomide may be related to the concomitant use of low molecular weight heparin (LMWH), capable of inhibiting PAR1 signaling on multiple myeloma cells. This is supported by the fact that the improved outcome in the patients treated with thalidomide in TT2 is almost entirely seen in HY disease with concomitant cytogenetic abnormalities (CA). The cytogenetic abnormalities, representing cells capable of proliferating in short term culture, in this group may be induced through PAR1-including the G-protein GNG11, a gene also overexpressed in and marking the unique form of HY disease (FIG. 7). FIGS. 8A-8B depict the correlation of DKK1 and GNG11 gene expression, respectively, in the seven molecularly defined multiple myeloma subtypes and healthy human plasma cells and human myeloma cell lines. Overexpression of DKK1 and GNG11 are greatest in the HY subtype of multiple myeloma.

The low level expression of CCND1 (FIG. 5), another target of β-catenin, that characterizes HY disease has also been difficult to explain. CCND1 expression in HY disease is activated by β-catenin signaling that is downstream of PAR1, not LRP5/6-frizzled receptors. The HY subtype is the only form of multiple myeloma that is not represented in multiple myeloma cell lines. This suggests that while this form of disease, representing over 50% of all multiple myeloma, can become highly aggressive, it remains perpetually dependent on signals from the bone microenvironment. According to the present invention, PAR1 signaling via thrombin signaling is this required component.

PAR1 Expression Correlates with EDNRB in Multiple Myeloma

One of the genes most highly correlated with PAR1 is EDNRB. PAR1 is known to activate EDNRB. In spite of EDNRB mapping to chromosome 13, which is frequently deleted in multiple myeloma, this gene is highly overexpressed in HY multiple myeloma to levels much greater than that seen in normal plasma cells. This implies that EDNRB is activated in HY multiple myeloma. The present invention shows that EDNRB activation in HY disease occurs through PAR1 activation. While this correlation is strong, it is not universal, such that some HY cases with high DKK1 lack expression of EDNRB and visa versa. Since PAR1 activation can trigger numerous signaling cascades, it is possible that this disconnect reflects the presence or absence of key signaling components required for the concomitant activation of these two genes in HY disease While the majority of cases have all the necessary components to link these two cascades, others will only have one.

### PAR1 Activation in Multiple Myeloma Induces Cell Quiescence

In-vitro studies indicate that activation of PAR1 in multiple myeloma cells induces cell quiescence. This finding would be consistent with a PAR1-mediated suppression of latent proliferation in focal lesions, while proliferation is unabated in vitro. It is likely that PAR1-induced quiescence contributes to the highly resistant, but low proliferative, phenotype of focal lesions in multiple myeloma. As such it follows that neutralizing PAR1 in multiple myeloma will have therapeutic benefit by increasing cell sensitivity to chemotherapy.

PAR1 is a G-protein coupled receptor. Experimental evidence suggests that PAR1 can induce numerous cell phenotypes, i.e. proliferation, differentiation. These differences are likely dependent on the type of G protein being expressed by the cells. Like PAR1 and DKK1, HY multiple myeloma is characterized by the overexpression of the G-protein. GNG11 is a member of the γ subunit family of heteromeric G-protein and is a potent inducer of cell senescence, e.g. quiescence. Thrombin-induced quiescence occurs in multiple myeloma cell lines that are positive for PAR1 and GNG11. Multicolor flow cytometry with CD38, CD45 and PAR1 antibodies has revealed that PAR1 positivity is associated with a more immature cell phenotype in both cell lines and primary tumor samples. This more immature cell phenotype within a given tumor is well recognized and the immature cells are thought to be cancer stem cells. Thus, PAR1 signaling in cells within focal lesions triggers a quiescence and de-differentiation, and may be critical to maintenance of minimal residual disease. PAR1 and DKK1 antagonism is expected to have synergistic effects. In appropriate PAR1 signaling in multiple myeloma and other cancers may be directly related to platelet activation and the release of thrombin in the vicinity of DKK1+/PAR1+ tumor masses. This leads to quiescence of the DKK1+/PAR1+ cells via GNG11. It would be therapeutically beneficial to inhibit PAR1 signaling using a PAR1 antagonist. The anticipated anti-myeloma effects might be related to Wnt/βcatenin but may involve many other signaling cascades.

### Example 3

### Levels of PAR1 Gene Expression are Associated with the Size of PAR1/F2R-Positive Subpopulations in HMCLs and Bone Marrow Aspirates

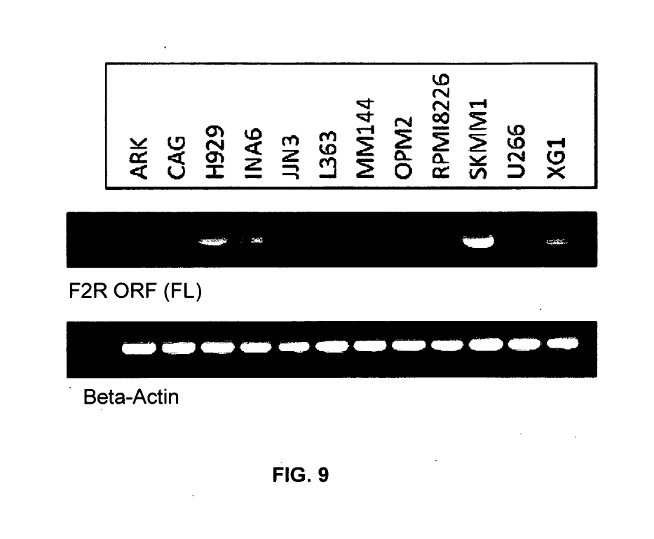

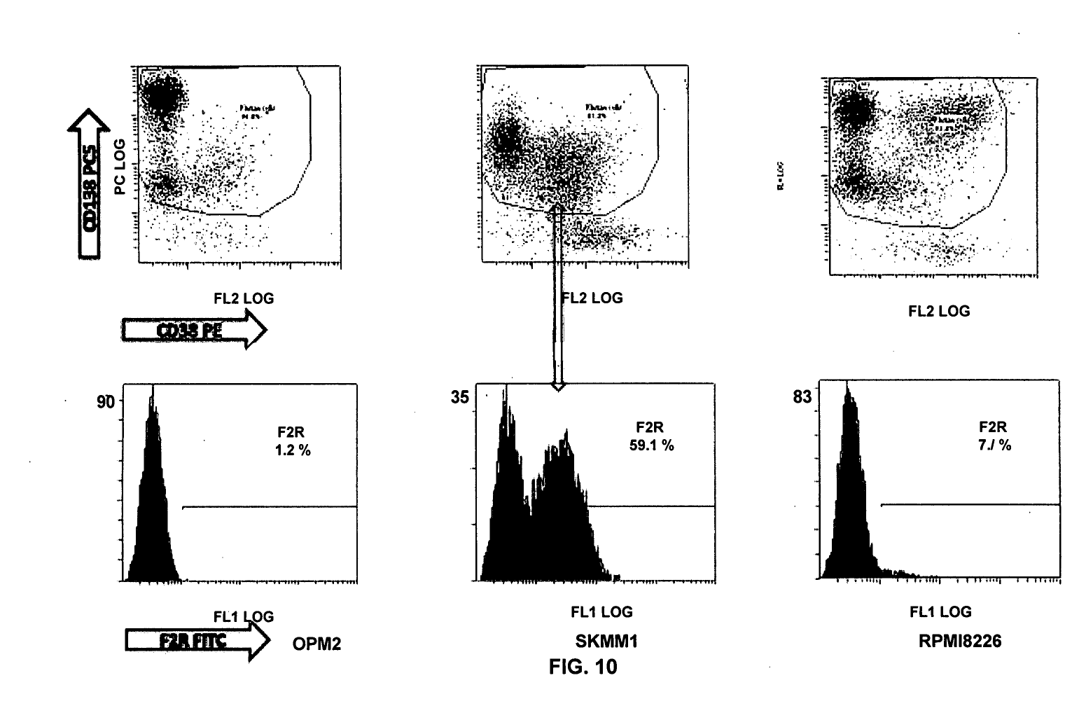

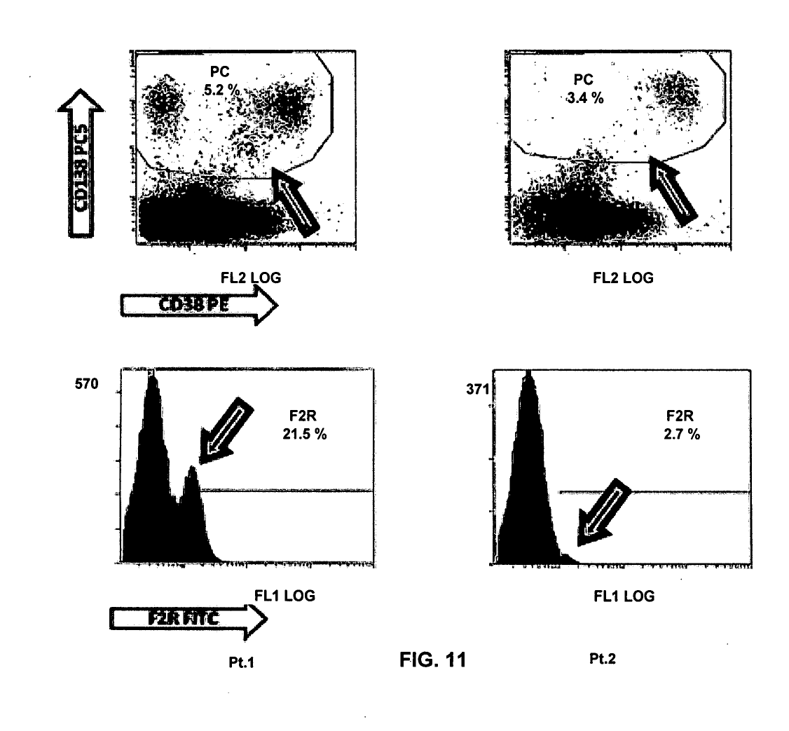

The full-length open reading frame of PAR1/F2R in a panel of HMCLs was measured using a semi-quantitative RT-PCR method (FIG. 9). The HMCLs demonstrated various levels of PAR1 expression. Using flow cytometry, it was found that not all the cells in a cell line express the PAR1 surface marker and that a subset of cells have the PAR1-positive phenotype combined with weak CD138 and CD38 expression (PAR1+/CD138dim/CD38dim) (FIG. 10). It was also demonstrated that the size of the PAR1+ subpopulation correlates with the quantitative levels of PAR1 gene expression in the cell lines and that cells expressing PAR1 represent a distinct population in a homogenous cell line. In primary myeloma bone marrow, PAR1+ cells were also detected with similar phenotypic markers as HMCLs (FIG. 11). It is contemplated that PAR1 expression is a tangible phenotype that allows precise characterization of a latent myeloma cell population which can be effective in identifying the myeloma stem cell.

### Immunomodulatory Drugs (IMiDs) Suppress Thrombin-Mediated PAR1 Signaling

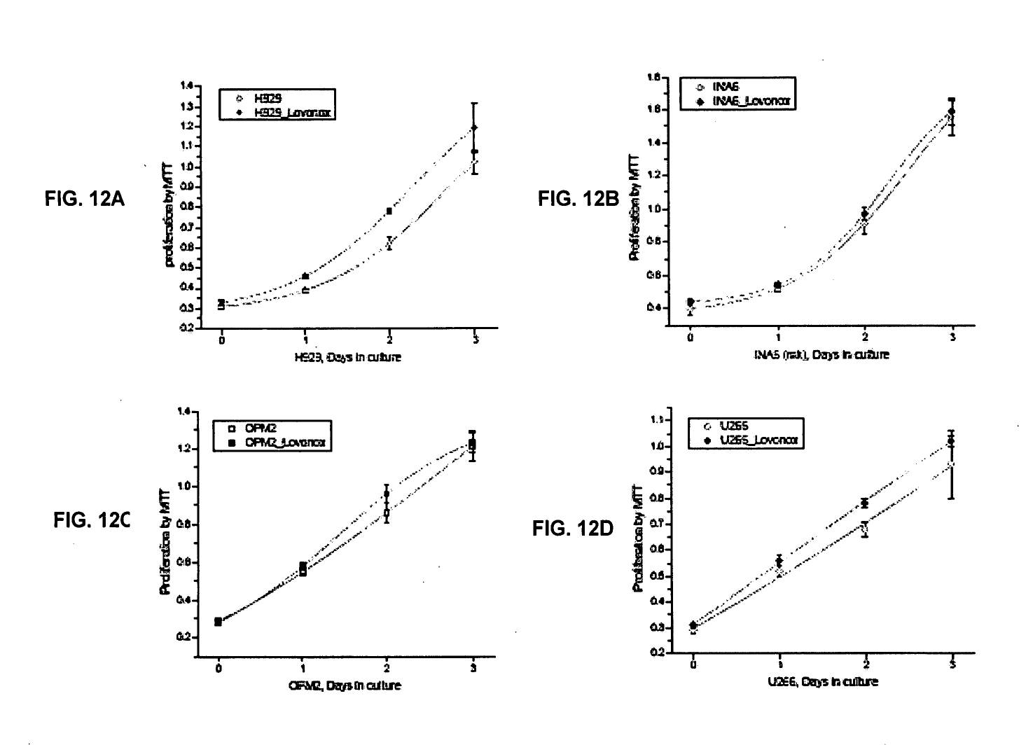

From a therapeutic point of view, the over expression of PAR1 may also explain another paradox observed in the long-term follow-up of patients treated with thalidomide in the TT2 trials where the beneficial effect of thalidomide is late emerging and can be seen even with its discontinuation because of toxicities. This effect may be related to the continued use of LMWH in these patients in spite of the discontinuation of thalidomide. What is interesting is that while human myeloma cell lines H929, INA5, OPM2 and U265 demonstrate slight increases in proliferation in the presence of the low molecular weight heparin LOVENOX, continued use of an LMWH may provide a therapeutic benefit during TT2 or other thalidomide therapies (FIGS. 12A-12D).

This phenomenon might have emerged only after the widespread adoption of the use of LMWH and other anti-deep vein thrombosis measures adopted by the community following the introduction of thalidomide use in 1999. This effect will not be seen in the pre thalidomide era and will be untraceable in the post thalidomide era as an immunomodulatory drug (ImiD) is now part of all treatments. As such, one of the effects of thalidomide may be to cause a massive initial degranulation of platelets within the first 48 hr of drug administration. This might also be linked to the wel-recognized phenomenon of DKK1 activation, possibly through PAR1-signaling.

It is possible that the immunomodulatory effects of the IMiDs is to suppress megakaryocyte function, platelets and therefore thrombin-mediated PAR1 signaling. Indeed, this may also be the mechanism by which lenalidomide exerts its anti-myelodysplastic syndrome (MDS) effects in myeloid stem cells form 5q-MDS. Whether lenalidomide is a potent anti-PAR1 signaling molecule and that combining these drugs with LMWH adds to the anti-thrombin signaling effects or whether the improved outcomes of the IMiDs is solely related to the co-administration of LMWH is not known.

The adverse side-effects seen with drugs may provide insights into their anti-tumor mechanisms of action. Therefore, the thrombocytopenia seen in some cases might have beneficial effects on disease with 5q gains, but not other types of disease. It is noteworthy that anecdotal evidence shows that following high dose melphalan, some of the only cells that remain are megakaryocytes. Thus, through their ability to shed platelets, which produce thrombin, megakaryocytes might be able to facilitate signaling through PAR1 on multiple myeloma cells and thereby contribute to the maintenance of minimal residual disease as well. Moreover, platelet infusions that are so often required following poly chemotherapy, and in particular high dose alkylator therapy, may have significant pro multiple myeloma growth effects in patients with PAR1 positive HY multiple myeloma, and especially those who do not receive IMiDs that might disrupt this signaling cascade. The CNV-dependent, plasma membrane receptor genes identified herein, including, but not limited to PAR1 represent potential biomarkers for use in GEP-based, flow cytometry or immunohistochemistry-based diagnostics and as therapeutic targets whose modulation may have anti-cancer activities.

### Example 4

### Thrombin Inhibits Proliferation of PAR1+ Myeloma Cells In Vitro

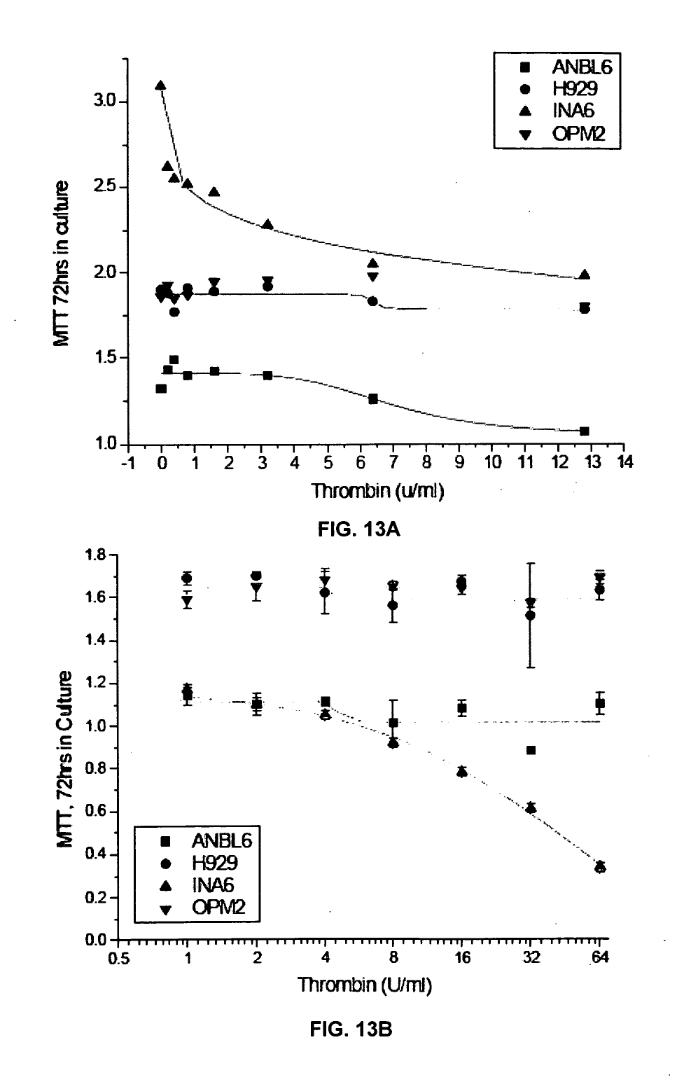

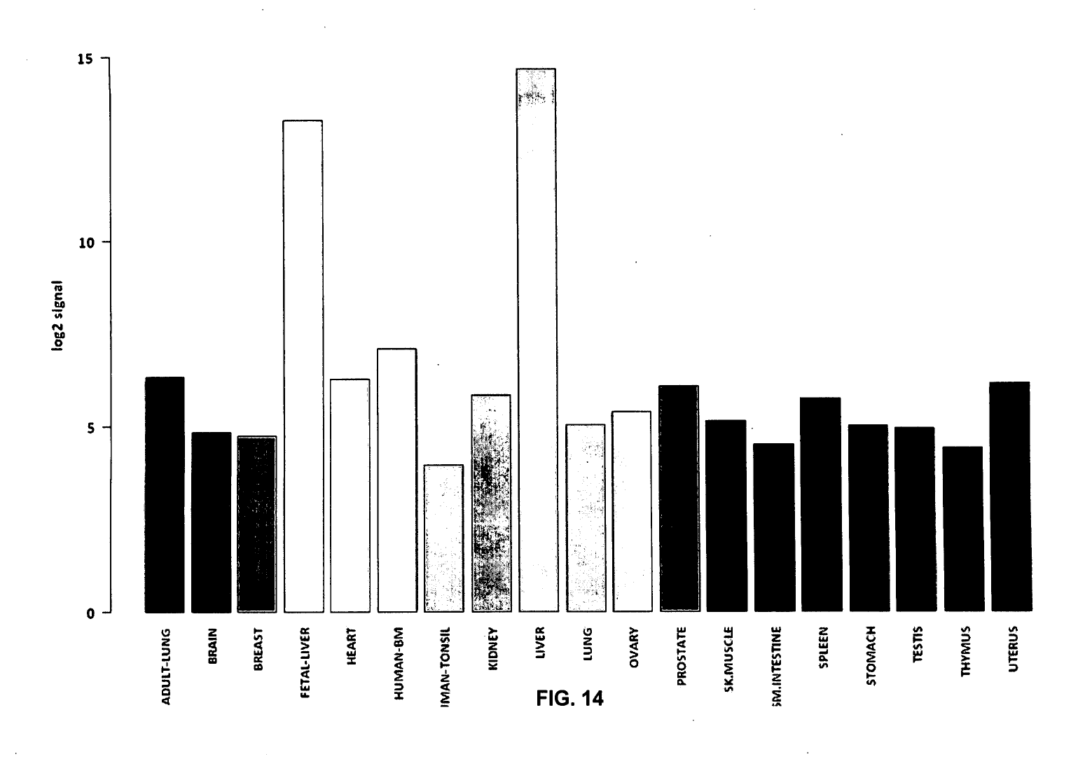

A panel of HMCLs that express PAR1 at high levels, e.g., H929 and INA6, and at low levels, e.g., OPM2, were exposed to sequentially titrated human thrombin. Growth inhibition was detected in the high-PAR1 cell lines but not in the OPM2 cell line (FIGS. 13A-13B). The results suggest thrombin has a regulatory effect in myeloma cell proliferation and that the PAR1 pathway is likely the responsive mechanism to thrombin. Myeloma cells with strong syndecan-1 expression and no PAR1 expression may not respond to thrombin same as the OPM2 cell line. Moreover, the high expression of PAR1/F2R in HY multiple myeloma and high thrombin production by the liver provides a reasonable explanation why HY myeloma tends to metasize to the liver (FIG. 14).

### Example 5

### Myeloma Cell Lines Express Various Levels of β-Catenin, and Drug-Resistance of Each Cell Line is Directly Linked to the Level of Intracellular β-Catenin

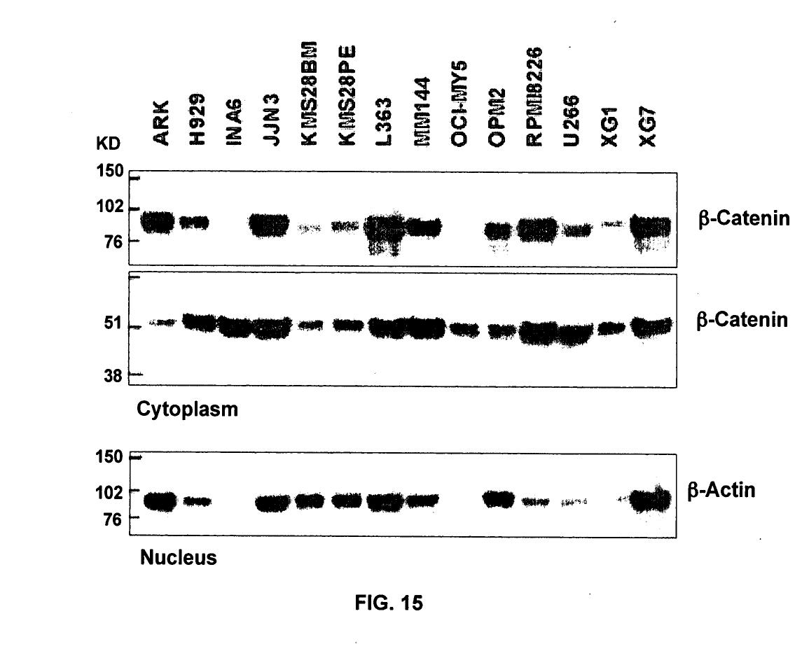

Protein was extracted from the cytoplasm and nucleus for Western Blot analyses and significantly diverse distributions of β-catenin were found in the fractions (FIG. 15). Cell growth inhibition of an anticancer agent (PU-H71) (6) was tested in a panel of myeloma cell lines. HMCLs were treated with a titration of the anti-cancer reagent PU-H71 (0 to 4,000 nM) in vitro for 72 hours. The MTT assay was utilized to detect proliferation of each cell line. The half maximal inhibitory concentration (IC50) of PU-H71 was calculated using OriginPro 7.5 statistic software. The IC50 correlates with the β-catenin protein levels in each cell line shown in FIG. 8. The results in Table 2 shows that cell lines with higher levels of β-catenin are more tolerant to higher doses of the drug.

TABLE 2

| Myeloma Cell Lines | IC50 of PU-H71 (nM) |
|---|---|
| ARK | 339.0 |
| H929 | 210.2 |
| INA6 | 141.2 |
| JJN3 | 799.8 |
| KMS28BM | 163.9 |
| KMS28PE | 343.7 |
| L363 | 231.6 |
| MM144 | 306.1 |
| OCI-MY5 | 163.9 |
| OPM2 | 414.7 |
| RPMI8226 | 485.0 |
| U266 | 197.6 |
| XG1 | 238.4 |

It also was demonstrated previously that these myeloma cell lines responded differently to bortezomib (7). The drug titration was conducted without the presence of thrombin. The activation of the PAR1 pathway may induce cell quiescence; therefore, the sensitivity to anti-myeloma drugs may also increase or decrease in each myeloma cell line depending on the expression levels of PAR1.

### Example 6

### The PAR1 Pathway Regulates Intracellular Distribution of β-Catenin

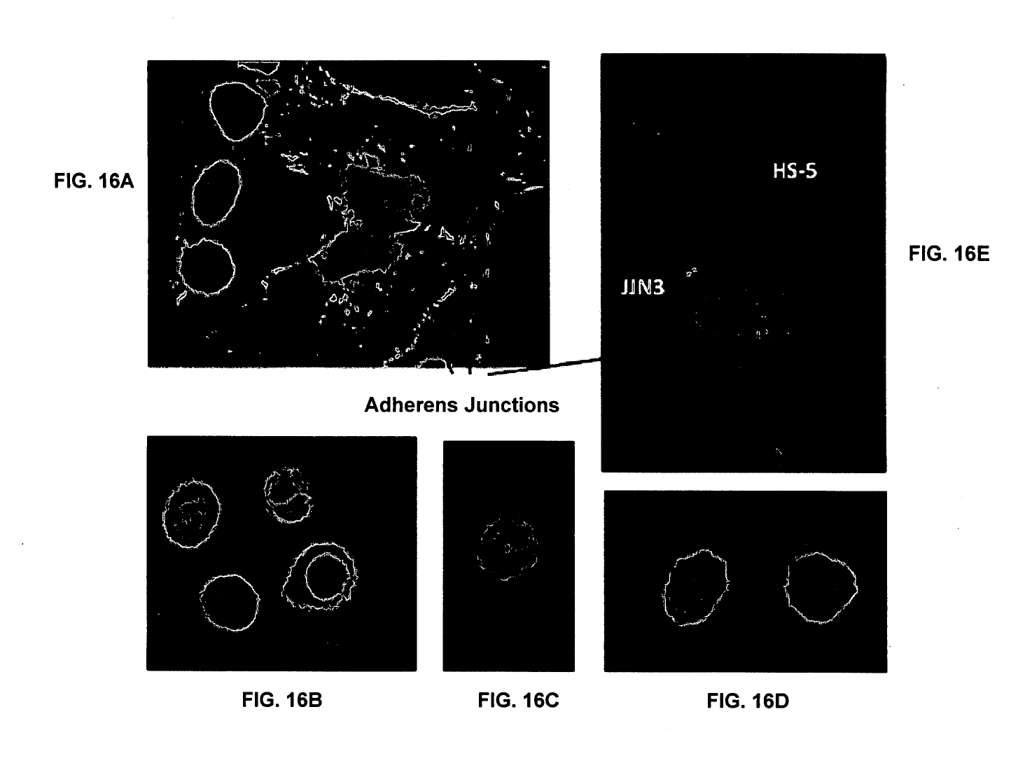

Unlike epithelial cells (FIG. 16A), myeloma cells do not show accumulation of β-catenin at the AJs of the cytoplasmic membrane. Instead, β-catenin primarily accumulates inside the nucleus (FIG. 16B). During the cell cycle, β-catenin may entirely redistribute to outside the nucleus when the cell is undergoing mitosis (FIG. 16C). At the end of mitosis, β-catenin is evenly distributed into the daughter cells and relocates back to the nucleus (FIG. 16D). In co-cultures of myeloma cells and stromal cells, AJs build up between myeloma and stromal cells, with intensified β-catenin presentation (FIG. 16E), but not between myeloma cells. Adding thrombin can increase myeloma cell-stromal cell affiliation.

### Example 7

### Characterization of a Subset of Myeloma Cells Expressing PAR1/F2R and have Myeloma Stem Cell Features

Gene expression profiles, clonal expansion, cytogenetics, and pharmacokinetic sensitivities of precisely selected PAR1+/CD138dim/CD38dim myeloma cells are compared to their with fully differentiated myeloma cell counterparts (PAR1−/CD138++/CD38++). Stromal co-cultures and SCID mouse models are utilized to imitate the microenvironment that supports the growth of primary tumor cells. Elevated PAR1 expression should be a marker that indicate the progression of primary myeloma cells to the quiescent stage. As membrane-bound β-catenin levels increase, myeloma cell quiescence occurs as well as more cell adhesion between myeloma cells and stromal cells. The role of PAR1-G protein coupled pathways as well as the Wnt pathway in the regulation of β-catenin redistribution in PAR1+/CD138dim/CD38dim cells is examined. It is contemplated that the intracellular redistribution of β-catenin drives the transformation of myeloma cells into the active stage and vice versa. Particularly, thrombin-induced PAR1 signaling modulates β-catenin redistribution and plays a major role in the reversible transformation (plasticity) of primary myeloma cells to a quiescent stem-like phenotype.

Selection of Cells with PAR1+/CD138dim/CD38dim Phenotype

Fifteen pairs of newly diagnosed or relapsed myeloma samples are collected and sorted for the PAR1 marker. HMCLs and primary myeloma specimens are subjected to PAR1-guided flow cytometric selection and cell sorting. Cells with the PAR1+/CD138dim/CD38dim phenotype are analyzed for their gene expression signature, proliferation index, cytogenetics, gene silencing/transfection alterations, and pharmacokinetic sensitivities and compared with their fully differentiated myeloma cell counterparts (PAR1-CD138++/CD38++). The in vitro growth microenvironment is established in a stromal cell co-culture system with human stromal cell lines (HS-5 or HS-27 from ATCC). Concurrently, in vivo, a SCID-human/rabbit mouse model, with an implantation of human or rabbit bone, is engrafted with the purified subset of myeloma cells for clonal expansion to expand the selected myeloma cell populations side by side.

Stabilization and Intracellular Distribution of B-Catenin in PAR1+/CD138dim/CD38dim Cells

β-catenin is examined as a functional protein in the transformation of myeloma cells into the quiescent stage. Using PAR1+/CD138dim/CD38dim cells, stabilization and intracellular distribution of β-catenin modulated by the PAR1-G protein-DVL axis as well as the Wnt pathway is examined. Thrombin and Wnt ligands, i.e. Wnt3A, Wnt5a, activate these pathways respectively. G-proteins, such as GNG11 and Gγ13, are up-regulated in myeloma cells and may involve in the PAR1 signal transduction. Gene silencing of PAR1 is done to knock out the gene transcripts in the PAR1+ cells. Stable gene silencing of PAR1 is essential for understanding the signaling pathways in control of transformation of myeloma cells to the quiescent stage through redistribution of β-catenin.

### Drug Resistance

Myeloma cells with the PAR1 phenotype are tested for drug resistance both in vitro and in vivo. Molecular targeting reagents are obtained by screening the inventory of the NCI and from pharmaceutics companies (Merck, Synta). PAR1+/CD138dim/CD38dim cells are treated with anti-myeloma reagents with or without co-culture systems to detect changes in the IC50. PAR1 antagonists are tested in SCID mice xenografted with primary tumor cells for pre-clinical validation of molecular targeting effects

The following references are cited herein.
1. Jiang, H. and Chess, L. J. Clin Invest, 2004, 114 9):1198-1208.
2. Peacock et al. PNAS Mar. 6, 2007, 104(10):4048-53.
3. Neuberger, M. S, and Milstein C. Curr Opin Immunol 1995, 7: 248.
4. Moon et al. Nat Rev Genet 2004, 691-701.
5. Zhan et al. Blood 2006, 108:2020-28.
6. Usmani et al. J. of Hem & One 2010, 3:40.
7. Slifka M K and Ahmed R. Lymphocyte activation and effector functions 252-258.
8. Smith et al. Eur J Immunol 1996, 26:444-448.
9. Matsui et al. Blood 15 Mar. 2004, 103(6):2332-36.
10. Laskowskia et al. Neuroscience Letters, 19 Mar. 2007, 415(1):17-21.
11. Hossaina et al. Bioch & Biophy Res Comm 2006, 351:645-50.
12. Reya T and Clevers H. Nature Apr. 14, 2005, 434:843-50.
13. Müller et al. J Biol Chem. Apr. 9, 1999, 274(15):10173-83.
14. Turm et al. J Biol Chem. May 14, 2010, 285(20):15137-48.
15. Tian et al. Mol Cancer Ther March 2008, 7:500.
16. Yaccoby et al. Blood 1998 Oct. 15, 92(8):2908-13.
17. Li et al. Br J Haem September 2007, 138 (6):802-11.
18. Zhan et al. Blood, 2003 Feb. 1, 101(3):1128-40.

1. A method for detecting a cytogenetic abnormality associated with multiple myeloma (MM) or monoclonal gammopathy of undetermined significance (MUGS) in a subject, comprising the steps of:
obtaining an isolated nucleic acid sample from an isolated biological sample from the subject;
determining the expression levels of copy number variant-dependent (CNV) genes PAR1, IL6R, IGF2R, GPR89A, EPHB1, GPR180, IL10RB, EGFR, DDR2, CCRL2, ADRB2, ADORA2A, GPR137B, CHRNA5, S1PR3, GPR146, GABRB3, PAQR6, HMMR, PTPRN2, MET, ADIPOR2, NCR1/p46NK, GPR175, and NPR3; and
comparing the expression levels of the CNV genes in the sample with expression levels of the CNV genes in a control sample; wherein one or both of an overexpression or an underexpression of the CNV genes as a result of copy number changes compared to control indicates the presence of a cytogenetic abnormality associated with MM or MUGS.

2. The method of claim 1, further comprising:
administering a pharmacological amount of at least one therapeutic agent effective to downregulate one or more of the overexpressed CNV genes or gene products or to upregulate one or more of underexpressed genes or gene products or a combination thereof, wherein altering expression of the one or more genes or gene products treats the MM or MUGS.

3. The method of claim 2, wherein PAR1 is downregulated in a multiple myeloma, the method further comprising simultaneously inhibiting DKK1 signaling.

4-5. (canceled)

6. The method of claim 1, wherein the overexpressed CNV genes are PAR1 or IL6R.

7. The method of claim 1, wherein PAR1 is overexpressed and PAR2, PAR3 and PAR4 are underexpressed.

8-9. (canceled)

10. The method of claim 1, wherein said multiple myeloma is a molecular subtype defined as hyperdiploid myeloma (HY).

11. The method of claim 1, wherein the biological sample is bone marrow or plasma cells.

12-30. (canceled)

31. A method for treating a multiple myeloma in a subject, comprising the step of:
administering a pharmacological amount of a therapeutic agent effective to inhibit the expression of one or more copy number variant-dependent (CNV) genes or gene products thereof, said genes comprising PAR1, IL6R, IGF2R, GPR89A, EPHB1, GPR180, IL10RB, EGFR, DDR2, CCRL2, ADRB2, ADORA2A, GPR137B, CHRNA5, S1PR3, GPR146, GABRB3, PAQR6, HMMR, PTPRN2, MET, ADIPOR2, NCR1/p46NK, GPR175 or NPR3 in either of a membrane or a soluble form, thereby treating the multiple myeloma.

32. The method of claim 31, wherein the CNV gene is PAR1, the method further comprising simultaneously administering a pharmacological amount of another therapeutic agent effective to inhibit DKK1 signaling.

33. The method of claim 31, wherein the therapeutic agent is a nucleic acid, a small molecule or an antibody.

34. The method of claim 31, wherein the multiple myeloma is a molecular subtype defined as hyperdiploid myeloma (HY).

35. The method of claim 31, further comprising the step of:
administering a pharmacological amount of a therapeutic agent effective to downregulate the expression of the PAR1 gene or an activity of a gene product thereof, thereby treating the multiple myeloma.

36. The method of claim 35, further comprising administering a pharmacological amount of a therapeutic agent effective to upregulate the expression of PAR2 and PAR3 genes or activities of gene products thereof.

37. The method of claim 35, further comprising administering a pharmacological amount of one or more anti-cancer drugs effective to treat the multiple myeloma, wherein the anti-cancer drug is melphalan, doxorubicin, cytoxan, etoposide, an IMiD, a proteasome inhibitor, or an HDAC inhibitor.

38-40. (canceled)

41. A method for treating a multiple myeloma in a subject, comprising the step:
administering an amount of a therapeutic agent pharmacologically effective to reduce megakaryocyte growth, thereby treating the multiple myeloma.

42. The method of claim 41, wherein the therapeutic agent is an inhibitor of expression or activity of one or both of MRVI1 or DKK1 gene or gene product.

43. The method of claim 41, wherein the therapeutic agent is thalidomide, lenalidomide, or a structural or functional derivative or an analogue thereof.

44-45. (canceled)

46. A method for lowering drug resistance in multiple myeloma cells, comprising the step of:
administering, one or more times, a pharmacological amount of a therapeutic agent effective to inhibit one or both of platelet activation or thrombin release in the multiple myeloma cells, wherein inhibition induces said multiple myeloma cells out of a state of quiescence, thereby lowering drug resistance in the cells.

47. The method of claim 46, wherein the therapeutic agent is an inhibitor of one or both of expression or activity of PAR1, GNG11, DKK1, or Wnt-B-catenin.

48-49. (canceled)

50. A method for increasing survivability of a subject with a multiple myeloma, comprising the step:
administering, one or more times, a pharmacological amount of a therapeutic agent effective to increase β-catenin gene expression or an activity of β-catenin protein, thereby increasing survivability of the subject.

51-52. (canceled)

53. The method of claim 31, wherein the subject was previously diagnosed as having multiple myeloma by the method of claim 1.

54. The method of claim 43, further comprising administering low molecular weight heparin to the subject.

55. The method of claim 2, wherein the treatment is treatment of MM and comprises reducing invasiveness, inhibiting metastasis to other tissues, inhibiting uncontrolled proliferation of malignant cells, or a combination thereof.
